(19) United States
(12) Patent Application Publication
(10) Pub. No.: US 2021/0361566 A1
(43) Pub. Date: Nov. 25, 2021

(54) FORMULATIONS OF 5-HYDROXY TRYPTOPHAN (5-HTP) FOR BETTER BIOAVAILABILITY FOR VARIOUS INDICATIONS

(71) Applicants: National University of Singapore, Singapore (SG); National University Hospital (Singapore) PTE LTD, Singapore (SG); Nanyang Technological University, Singapore (SG)

(72) Inventors: Jacob Pade Ramsoe JACOBSEN, Durham, NC (US); Johnson FAM, Singapore (SG); Subramanian VENKATRAMAN, Singapore (SG)

(21) Appl. No.: 17/252,961

(22) PCT Filed: Jun. 14, 2019
(86) PCT No.: PCT/US2019/037349

§ 371 Date: Dec. 16, 2020

Related U.S. Application Data
(60) Provisional application No. 62/686,774, filed on Jun. 19, 2018.

(51) Int. Cl. A61K 9/00; A61K 31/405; A61K 45/06; A61K 9/20

(52) U.S. Cl. CPC A61K 9/0065; A61K 31/405; A61K 9/2077; A61K 9/2054; A61K 45/06

(57) ABSTRACT

Disclosed herein are a range of gastroretentive sustained release dosage forms that may be particularly useful in the delivery of 5-HTP and other agents that would benefit from delivery to the upper gastrointestinal tract.

### CROSS REFERENCE TO RELATED APPLICATIONS

This application claims priority to and the benefit of U.S. Provisional Patent Application Ser. No. 62/686,774, filed Jun. 19, 2018; the disclosure of which is incorporated herein by reference in its entirety.

### TECHNICAL FIELD

The present invention relates to a pharmaceutical composition with a sustained release (SR) profile. In particular it relates to a pharmaceutical composition comprising a therapeutically effective amount of 5-hydroxytryptophan (5-HTP) with a SR profile. Still further, it relates to a gastroretentive pharmaceutical composition with a SR profile. The pharmaceutical composition may be effective in treating conditions selected from depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's, Parkinson's), stroke recovery, autism, migraine, sleep disorders, premenstrual dysphoria, post-traumatic stress disorder, post-partum depression, phenylketonuria, and depression after interferon treatment.

### BACKGROUND

The listing or discussion of an apparently prior-published document in this specification should not necessarily be taken as an acknowledgement that the document is part of the state of the art or is common general knowledge.

Drugs that inhibit the serotonin transporter, such as Selective Serotonin Reuptake Inhibitors (SSRIs), Serotonin-Noradrenaline Reuptake Inhibitors (SNRIs) and certain members of the Tricyclic Antidepressant (TCA) class of drugs, are currently used in the treatment of several CNS disorders, including depressive and anxiety disorders. SSRIs, for example, are believed to increase the extracellular level of the neurotransmitter serotonin (also known as 5-hydroxytryptamine, 5-HT) by limiting its reabsorption into the presynaptic and postsynaptic neurons. This increases the level of serotonin available to bind to the postsynaptic and presynaptic serotonin receptors. This, in turn, is believed to elicit neurobiological changes that over time produces the therapeutic response (e.g. see Blier, Pierre, and Claude De Montigny. “Current advances and trends in the treatment of depression.” Trends in pharmacological sciences (1994), 15(7): 220-226).

Among the alternatives to the drugs currently used in the treatment of CNS disorders is 5-hydroxytryptophan (5-HTP). 5-HTP is the immediate precursor of serotonin. In preliminary studies, 5-HTP has been reported to have some clinical efficacy in depression (Turner, Erick H., Jennifer M. Loftis, and Aaron D. Blackwell. “Serotonin a la carte: supplementation with the serotonin precursor 5-hydroxytryptophan.” Pharmacology & therapeutics (2006), 109(3): 325-338) and in other CNS indications (Birdsall T C. “5-Hydroxytryptophan: a clinically-effective serotonin precursor.” Altern Med Rev. (1998), 3(4):271-80. Review. PubMed PMID: 9727088). The elimination half-life of 5-HTP is about 1.5-2 hours, which is too short for practical clinical use, but can be increased to up to four hours when co-administered with a high dose of a peripheral decarboxylase inhibitor, such as carbidopa or benserazide. See U.S. Pat. No. 8,969,400. Peripheral decarboxylase inhibitors inhibit the conversion of 5-HTP to serotonin, but only outside the brain, as peripheral decarboxylase inhibitors cannot cross the blood-brain barrier. Overall, however, the pharmacokinetics of 5-HTP can limit the utility of immediate-release formulations of 5-HTP due to large fluctuations in the plasma levels of 5-HTP, making multiple daily doses necessary and/or giving rise to the possibility of alternating overdosing, causing side effects, and underdosing, causing intermitting loss of therapeutic efficacy (Jacobsen, Jacob P R, et al. “Adjunctive 5-Hydroxytryptophan slow-release for treatment-resistant depression: clinical and preclinical rationale.” Trends in pharmacological sciences (2016), 37(11): 933-944). As described in U.S. Pat. No. 8,969,400, 5-HTP treatment in immediate-release formulations has also been associated with gastrointestinal adverse events in some patients.

Accordingly, there remains a need for additional formulations for 5-HTP, particularly for additional sustained release formulations for 5-HTP.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a gastroretentive sustained release (SR) dosage form comprising 5-hydroxytryptophan (5-HTP) or a pharmaceutically acceptable salt or solvate thereof and a pharmaceutically acceptable carrier and/or excipient, wherein the dosage form provides a release rate to the upper gastrointestinal tract of between about 2.5 milligrams per hour (mg/hr) and about 75 mg/hr, thereby providing a steady state plasma level of between about 0.1 milligrams per liter (mg/L) to about 4 mg/L at steady state.

In some embodiments, the dosage form comprises at least a first polymeric matrix material that swells in the presence of gastric fluid, thereby providing a swellable dosage form that increases in size to promote retention of the dosage form in the stomach, optionally wherein the dosage form swells in the presence of gastric fluid to at least about 150% compared to a pre-swelling volume of the dosage form. In some embodiments, the first polymeric matrix material comprises a hydrophilic polymer selected from the group consisting of polyoxyethylene oxide, hydroxyethylcellulose, carboxymethylcellulose, polyethylene glycol diacrylate (PEGDA), gelatin, gelatin-PEGDA copolymer, hyaluronic acid, chitosan, hydroxypropylcellulose, hydroxypropylmethylcellulose, sodium acrylate, and copolymers thereof. In some embodiments, the 5-HTP or pharmaceutically acceptable salt or solvate thereof is directly dispersed in the first polymeric matrix material in an amount between about 1 weight % (wt %) and about 50 wt % based on the weight of the first polymeric matrix material.

In some embodiments, the dosage form further comprises: a plurality of microparticles dispersed within the first polymeric matrix material, wherein each of said microparticles comprises a second polymeric matrix material and 5-HTP or a pharmaceutically acceptable salt or solvate thereof dispersed within the second polymeric matrix material, and wherein the first polymeric matrix material comprises 5-HTP or a pharmaceutically acceptable salt or solvate thereof directly dispersed in the first polymeric matrix material in an amount between about 0 wt % and about 50 wt % based on the weight of the first polymeric matrix material. In some embodiments, the second polymeric matrix material comprises: a crosslinked polymeric matrix material comprising one or more hydrophilic polymer selected from the group consisting of hydroxyl propyl methyl cellulose, hydroxyl propyl cellulose, hyaluronic acid, chitosan, gelatin, gelatin-PEGDA, PEGDA, and sodium acrylate; and/or a non-crosslinked polymeric matrix material comprising one or more hydrophilic polymer selected from the group consisting of chitosan, poly(ethylene oxide), hydroxyl propyl cellulose and hydroxypropyl methylcellulose.

In some embodiments, the first polymeric matrix material contains between about 5 wt % and about 50 wt % of the microparticles. In some embodiments, each microparticle comprises between about 1 wt % and about 30 wt % of 5-HTP or a pharmaceutically acceptable salt or solvate thereof based on the weight of the microparticle.

In some embodiments, the dosage form comprises between about 50 milligrams (mg) and about 1,800 mg of 5-HTP or a pharmaceutically acceptable salt or solvent thereof. In some embodiments, at least about 30 weight % (wt %) of the 5-HTP or pharmaceutically acceptable salt or solvate thereof is released within about 4 hours of oral administration, optionally wherein at least about 50 wt % of the 5-HTP or pharmaceutically acceptable salt or solvate thereof is released within about 4 to about 9 hours of oral administration.

In some embodiments, the dosage form further comprises one or more additional agent selected from the group consisting of a serotonin-enhancing compound, a peripheral decarboxylase inhibitor, and a gas swelling agent. In some embodiments, the dosage form is adapted to deliver a release profile of between about 1 mg/hr and about 42 mg/hr of 5-HTP for a period of about 12 hours, optionally wherein the release profile is substantially linear. In some embodiments, the dosage form provides a release rate to the upper gastrointestinal tract of about 6.25 mg/hr, so as to provide an average steady state 5-HTP plasma level of about 0.25 mg/L.

In some embodiments, the presently disclosed subject matter provides a method of treating a condition selected from the group consisting of depression, social anxiety, panic disorder, generalized anxiety disorder, OCD, impulse control disorders, suicidality, borderline personality disorder, fibromyalgia, ataxia, mood symptoms and agitation related to neurological disorders, stroke recovery, autism, migraine, sleep disorders, premenstrual dysphoria, post-traumatic stress disorder, post-partum depression, phenylketonuria, and depression after interferon treatment in a patient in need of such treatment. In some embodiments, the method comprises administering a dosage form in accordance with the presently disclosed subject matter. In some embodiments, the dosage form is administered once or twice daily. In some embodiments, the dosage form is administered with a meal.

In some embodiments, the dosage form is administered once or twice daily and the total amount of 5-HTP in the daily dosage is between about 50 mg and about 3600 mg. In some embodiments, the dosage form is adapted to deliver a release profile of between about 4 mg/hr and about 42 mg/hr of 5-HTP for a period of about 12 hours, optionally wherein the release profile is substantially linear. In some embodiments, administration of the dosage form provides a steady state 5-HTP plasma level of between about 0.1 mg/L and about 0.9 mg/L.

In some embodiments, the method further comprises concomitant administration of a 5-HTP absorption enhancer to increase the steady state 5-HTP plasma level between about 1-fold and about 4-fold as compared to when the 5-HTP is administered without the absorption enhancer, optionally wherein the 5-HTP absorption enhancer is a peripheral decarboxylase inhibitor.

In some embodiments, a method of achieving a steady state 5-HTP plasma level of between about 0.1 mg/L to 1 mg/L is provided. In some embodiments, the method comprises administering between about 2.5 mg/hr and about 25 mg/hr of 5-HTP or a pharmaceutically acceptable salt or solvate thereof to the upper gastrointestinal tract. In some embodiments, the method achieves a steady state 5-HTP plasma level of about 0.25 mg/L by administering about 6.25 mg/hr of 5-HTP or a pharmaceutically acceptable salt or solvate thereof to the upper gastrointestinal tract.

Accordingly, it is an object of the presently disclosed subject matter to provide gastroretentive, sustained release formulations for 5-HTP.

An object of the presently disclosed subject matter having been stated hereinabove, and which is achieved in whole or in part by the presently disclosed subject matter, other objects will become evident as the description proceeds when taken in connection with the accompanying drawings and examples as best described herein below.

### BRIEF DESCRIPTION OF THE DRAWINGS

In order that the disclosure may be readily understood and put into practical effect, reference will now be made to examples as illustrated with reference to the accompanying figures. The figures together with the description serve to further illustrate the embodiments of the invention and explain various principles and advantages.

FIG. 1 is a plot of plasma concentration (in nanograms per milliliter (ng/ml)) of 5-hydroxytryptophan (5-HTP) versus time (in hours (h)) for oral (200 milligram (mg), circles), colonic (200 mg, squares) and intravenous (IV, 50 mg, triangles) administration of 5-HTP in human volunteers.

FIG. 2A is a schematic diagram showing the entry of a gastroretentive sustained release (SR) 5-hydroxytryptophan (5-HTP) formulation that can achieve the release profile of the presently disclosed subject matter into the stomach. The formulation comprises a first polymer matrix (indicated by the oval) with microparticles (circles) dispersed within the first polymer matrix. The microparticles comprise a second polymer matrix with 5-HTP (“X”s) dispersed therein. In some embodiments, a peripheral decarboxylase inhibitor (e.g., carbidopa or benserazide) can also be included in the microparticles and/or first matrix material to decrease degradation of 5-HTP in the intestine and thus enhance absorption and bioavailability of the 5-HTP.

FIG. 2B is a schematic diagram showing the formulation described for FIG. 2A undergoing swelling in the gastric fluids in the stomach so that the formulation becomes too large to pass into the intestine.

FIG. 2C is a schematic diagram showing the formulation described for FIGS. 2A and 2B where the microparticles (circles) are releasing 5-hydroxytryptophan (5-HTP, “X”s) into the swollen first matrix material (indicated by the oval).

FIG. 2D is a schematic diagram showing the formulation described for FIGS. 2A-2C where 5-hydroxytryptophan (5-HTP, “X”s) and 5-HTP-containing microparticles (circles) are diffusing from the swollen first matrix material (indicated by the oval) into the gastric fluids in the stomach and upper intestine.

FIG. 3A is a schematic diagram showing the entry of a gastroretentive sustained release (SR) 5-hydroxytryptophan (5-HTP) formulation that can achieve the release profile of the presently disclosed subject matter into the stomach. The formulation comprises a first polymer matrix (indicated by the oval) with microparticles (circles) dispersed within the first polymer matrix. The microparticles comprise a second polymer matrix with 5-HTP (“X”s) dispersed therein. In some embodiments, a peripheral decarboxylase inhibitor (e.g., carbidopa or benserazide) can also be included in the microparticles and/or first matrix material to decrease degradation of 5-HTP in the intestine and thus enhance absorption and bioavailability of the 5-HTP.

FIG. 3B is a schematic diagram showing the formulation described for FIG. 3A undergoing swelling in the gastric fluids in the stomach so that the formulation becomes too large to pass into the intestine.

FIG. 3C is a schematic diagram showing the formulation described for FIGS. 3A and 3B where the microparticles (circles) are releasing 5-hydroxytryptophan (5-HTP, “X”s) into the first matrix material (indicated by the oval).

FIG. 3D is a schematic diagram showing the formulation described for FIGS. 3A-3C where 5-hydroxytryptophan (5-HTP, “X”s) is diffusing from the first matrix material (indicated by the oval) into the gastric fluids in the stomach and upper intestine, while the microparticles stay in the first matrix material.

FIG. 4A is a schematic diagram showing the entry of a gastroretentive sustained release (SR) 5-hydroxytryptophan (5-HTP) formulation of the presently disclosed subject matter into the stomach. The formulation comprises a polymer matrix (indicated by the oval) with 5-HTP (“X”s) dispersed within the polymer matrix. In some embodiments, a peripheral decarboxylase inhibitor (e.g., carbidopa or benserazide) can also be included in the matrix material to decrease degradation of 5-HTP in the intestine and thus enhance absorption and bioavailability of 5-HTP.

FIG. 4B is a schematic diagram showing the formulation described for FIG. 4A undergoing swelling in the gastric fluids in the stomach so that the formulation becomes too large to pass into the intestine.

FIG. 4C is a schematic diagram showing the formulation described for FIGS. 4A and 4B where the 5-hydroxytryptophan (5-HTP, “X”s) is diffusing within the matrix material (indicated by the oval).

FIG. 4D is a schematic diagram showing the formulation described for FIGS. 4A-4C where 5-hydroxytryptophan (5-HTP, “X”s) is diffusing from the matrix material (indicated by the oval) into the gastric fluids in the stomach and upper intestine.

### DETAILED DESCRIPTION

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which the presently described subject matter belongs. All publications, patent applications, patents, and other references mentioned herein are incorporated by reference in their entirety.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “an agent” or “a polymer” includes a plurality of such agents or polymers, and so forth.

Unless otherwise indicated, all numbers expressing quantities of size, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of size (i.e., diameter), weight, concentration or percentage is meant to encompass variations of in one example ±20% or ±10%, in another example ±5%, in another example ±1%, and in still another example ±0.1% from the specified amount, as such variations are appropriate to perform the disclosed methods.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

The term “matrix”, as used herein, denotes its well-known meaning in the pharmaceutical arts, that is, a solid material, optionally having an active ingredient incorporated therein, providing swelling or structural support.

In pharmacokinetics and as used herein, “steady state” refers to the situation where the overall intake of an active pharmaceutical compound is fairly in dynamic equilibrium with its elimination. Thus, the average plasma level of the compound remains the same from day to day, although there can be intra-day fluctuations related to dosing. In practice, for most drugs, it typically takes between about 4 and about 6 half-lives to reach steady state after regular dosing is started.

### II. General Considerations

The upper intestinal transit time is around 3-4 hours (Hua S, Marks E, Schneider J J, Keely S. “Advances in oral nano-delivery systems for colon targeted drug delivery in inflammatory bowel disease: selective targeting to diseased versus healthy tissue.” Nanomedicine. (2015), 11(5):1117-32). Hence, conventional solid dosage forms orally ingested in the fasted state (e.g., standard tablets, capsules, particulates, etc.) quickly transit through the stomach and normally arrive in the colon after around 3-4 hours. Therefore, conventional sustained release (SR) technologies (e.g., such as those described in U.S. Pat. No. 7,670,619) require that the active compound is absorbed in the colon if the delivery profile extends beyond 3 hours, in order to deliver a therapeutically effective dose for the full SR delivery period.

For example, U.S. Pat. No. 7,670,619 describes a 5-HTP SR formulation comprising a double-layered tablet, one layer containing 5-HTP for fast release (“fast” layer) and the other layer containing tryptophan or 5-HTP for retarded release (“retard” layer). The manufacturing process for the double-layered tablets requires separate preparation of the two blends for the “fast” and “retard” layers followed by compression with an appropriate tableting device that ensures the separation, the integrity and release characteristics of each layer. The tablets can readily transition through the stomach, thus delivering the great majority of the 5-HTP in the upper and lower intestine (colon), for absorption in both the upper and lower intestine.

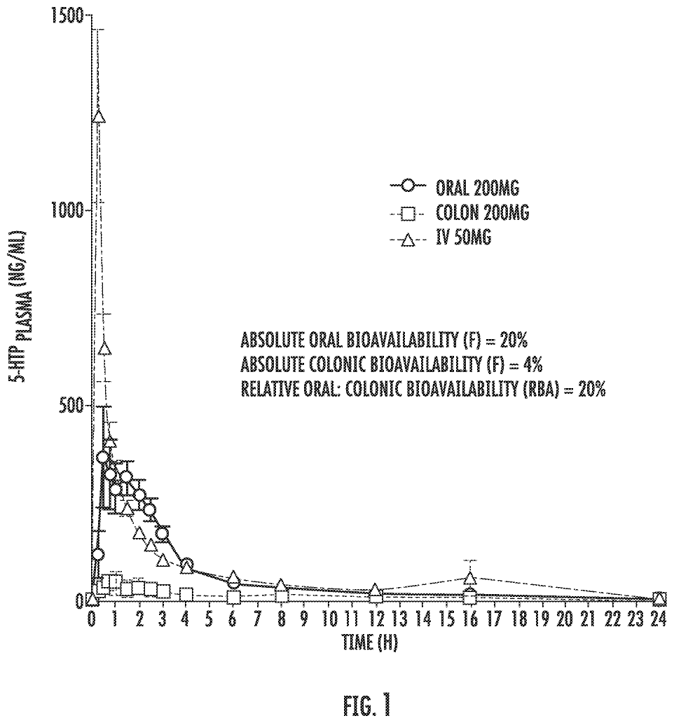

However, according to one aspect of the presently disclosed subject matter, it been demonstrated for the first time that 5-HTP is only minimally absorbed in the human colon. See FIG. 1 and Example 1, below. More particularly, as described in FIG. 1 and Example 1, 5-HTP has substantial bioavailability in humans in the upper intestine (F=20%), but not in the colon (F=4%). This data contrasts to findings in mice, where 5-HTP is very effectively absorbed in the colon (Jacobsen et al., Neuropsychopharmacology (2016), 41:2324-2334) and could not have been inferred from previously published data or teachings. Therefore, in one aspect, the presently disclosed subject matter is directed to 5-HTP formulations adapted for SR to the upper gastrointestinal (GI) tract, and, in particular, to gastroretentive SR formulation technologies. The presently disclosed subject matter further provides gastroretentive 5-HTP SR formulations for the treatment of human disorders. For example, the presently disclosed subject matter provides dosage forms adapted to remain in the stomach for several hours (e.g., up to about 12 hours) and that have particular 5-HTP release rates. Therefore, the dosage forms provide particular 5-HTP release rates to the upper GI tract over a period of several hours (e.g., up to about 12 hours).

In some embodiments, the presently disclosed subject matter provides SR formulations (e.g., swellable gastroretentive SR formulations) comprising two or more separate matrices incorporated in one dosage form. In some embodiments, the formulation can comprise a swellable gastroretentive matrix comprising microparticles of another matrix material.

In contrast to currently available dosage forms, the presently disclosed dosage forms remain in the stomach and delivers the majority (e.g., 80% or more) of the 5-HTP, and optionally other incorporated active ingredients, in the stomach and upper gastrointestinal tract for absorption exclusively in the upper intestine.

In some embodiments, the presently disclosed subject matter provides a gastroretentive SR dosage form, comprising 5-HTP or a pharmaceutically acceptable salt or solvate thereof, wherein:

the 5-HTP or a pharmaceutically acceptable salt or solvate thereof is present in an amount of from about 50 to about 1,000 mg;

at least about 30 wt % of the 5-HTP or pharmaceutically acceptable salts or solvates thereof is released within between about 3 hours and about 5 hours of oral administration; and

up to about 100 wt % of the 5-HTP or pharmaceutically acceptable salts or solvates thereof is released within between about 8 hours and about 12 hours of oral administration. In some embodiments, the dosage form can comprise more than one pharmaceutically acceptable salt and/or solvate of 5-HTP.

Additionally or alternatively, the presently disclosed subject matter provides a gastroretentive SR dosage form comprising 5-HTP or a pharmaceutically acceptable salt or solvate thereof and a pharmaceutically acceptable carrier and/or excipient, wherein the dosage form provides a release rate to the upper gastrointestinal tract of between about 2.5 mg/hr to about 25 mg/hr, so as to provide a steady state 5-HTP plasma level of between about 0.1 mg/L to about 1 mg/L (e.g. the dosage form may provide a release rate to the upper gastrointestinal tract of 6.25 mg/hr, so as to provide a 5-hydroxytryptophan plasma level of 0.25 mg/L). In some embodiments, the pharmaceutically acceptable carrier and/or excipient comprises a swellable hydrophilic polymeric matrix material.

Additionally or alternatively, the presently disclosed subject matter provides a gastroretentive SR dosage form comprising 5-HTP or a pharmaceutically acceptable salt or solvate thereof and a pharmaceutically acceptable carrier and/or excipient, wherein the dosage form provides a release rate to the upper gastrointestinal tract of between about 2.5 mg/hr to about 75 mg/hr, so as to provide a steady state 5-HTP plasma level of between about 0.1 mg/L to about 3 mg/L (e.g. the dosage form may provide a release rate to the upper gastrointestinal tract of about 6.25 mg/hr, so as to provide a steady state 5-HTP plasma level of about 0.25 mg/L; or a release rate to the upper gastrointestinal tract of about 12.5 mg/hr, so as to provide a steady state 5-HTP plasma level of about 0.5 mg/L). In some embodiments, the pharmaceutically acceptable carrier and/or excipient comprises a swellable hydrophilic polymeric matrix material.

### III. Sustained Release Dosage Forms and Related Methods and Uses

Thus, in a first aspect of the invention, there is provided a gastroretentive sustained release (SR) dosage form comprising 5-HTP or a pharmaceutically acceptable salt or solvate thereof, wherein:

the 5-HTP or pharmaceutically acceptable salt or solvate thereof is present in an amount of between about 50 to about 1,000 mg;

at least about 30 wt % of the 5-HTP or pharmaceutically acceptable salt or solvate thereof is released within between about 3 hours and about 5 hours of oral administration; and

up to about 100 wt % of the 5-HTP or pharmaceutically acceptable salt or solvate thereof is released between about 8 hours and about 12 hours of oral administration.

In certain embodiments:

(a) at least about 30 wt % of the 5-HTP or pharmaceutically acceptable salt or solvate thereof is released within about 4 hours of oral administration; and/or

(b) at least about 50 wt % of the 5-HTP or pharmaceutically acceptable salt or solvate thereof is released within between about 4 hours and about 9 hours of oral administration, such as within about 4, 5, 6, 7, 8, or 9 hours of oral administration; and/or

(c) at least about 80 wt % of the 5-HTP or pharmaceutically acceptable salt or solvate thereof is released within between about 6 hours and about 12 hours of oral administration, such as within between about 8 hours and about 12 hours of oral administration (e.g., within between about 8 hours, within about 9 hours, or within about 10 hours of oral administration).

In a second aspect of the invention, there is provided a dosage form comprising 5-HTP or a pharmaceutically acceptable salt or solvate thereof and a pharmaceutically acceptable carrier and/or excipient, wherein the dosage form provides a release rate of 5-HTP to the upper gastrointestinal (GI) tract of from between about 2.5 mg/hr and about 75 mg/hr, so as to provide a steady state 5-HTP plasma level of between about 0.1 mg/L and about 4 mg/L. In some embodiments, the release rate to the upper GI is relatively linear (i.e., wherein about the same amount of 5-HTP is released every hour for up to about 6, 7, 8, 9, 10, 11, or about 12 hours or more). In some embodiments, the dosage form provides a steady state 5-HTP plasma level of between about 0.1 mg/L and about 3 mg/mL. In some embodiments, the dosage form provides a release rate of 5-HTP to the upper GI tract of between about 2.5 mg/hr and about 25 mg/hr (e.g., about 2.5, about 5.0, about 7.5, about 10, about 12.5, about 15, about 20, or about 25 mg/hr), so as to provide a steady state 5-HTP plasma level of between about 0.1 mg/L and about 1 mg/L (e.g., about 0.1, 0.2, 0.3, 0.4, 0.5, 0.6, 0.7, 0.8, 0.9, or about 1.0 mg/L).

More particularly, in some embodiments, the dosage form provides a release rate of 5-HTP to the upper GI tract of about 2.5 mg/hr, so as to provide an average steady state plasma level of 5-HTP of about 0.10 mg/L. In some embodiments, the dosage form provides a release rate of 5-HTP to the upper GI tract of about 5.0 mg/hr, so as to provide an average steady state plasma level of 5-HTP of about 0.20 mg/L. In some embodiments, the dosage form provides a release rate of 5-HTP to the upper GI tract of about 7.5 mg/hr, so as to provide an average steady state plasma level of 5-HTP of about 0.30 mg/L. In some embodiments, the dosage form provides a release rate of 5-HTP to the upper GI tract of about 10 mg/hr, so as to provide an average steady state plasma level of 5-HTP of about 0.40 mg/L. In some embodiments, the dosage form provides a release rate of 5-HTP to the upper GI tract of about 12.5 mg/hr, so as to provide an average steady state plasma level of 5-HTP of about 0.50 mg/L. In some embodiments, the dosage form provides a release rate of 5-HTP to the upper GI tract of about 15 mg/hr, so as to provide an average steady state plasma level of 5-HTP of about 0.60 mg/L. In some embodiments, the dosage form provides a release rate of 5-HTP to the upper GI tract of about 17.5 mg/hr, so as to provide an average steady state plasma level of 5-HTP of about 0.70 mg/L. In some embodiments, the dosage form provides a release rate of 5-HTP to the upper GI tract of about 20 mg/hr, so as to provide an average steady state plasma level of 5-HTP of about 0.80 mg/L. In some embodiments, the dosage form provides a release rate of 5-HTP to the upper GI tract of about 22.5 mg/hr, so as to provide an average steady state plasma level of 5-HTP of about 0.90 mg/L.

In some embodiments, the steady state 5-HTP plasma level is increased about 1-fold by concomitant administration of a 5-HTP absorption enhancer as compared to when the 5-HTP is administered without the absorption enhancer. In some embodiments, the steady state 5-HTP plasma level is increased about 2-fold by concomitant administration of a 5-HTP absorption enhancer as compared to when the 5-HTP is administered without the absorption enhancer. In some embodiments, the steady state 5-HTP plasma level is increased about 3-fold by concomitant administration of a 5-HTP absorption enhancer as compared to when the 5-HTP is administered without the absorption enhancer. In some embodiments, the steady state 5-HTP plasma level is increased about 4-fold by concomitant administration of a 5-HTP absorption enhancer as compared to when the 5-HTP is administered without the absorption enhancer. Thus, in some embodiments, the dosage form comprises a 5-HTP absorption enhancer. In some embodiments, the 5-HTP absorption enhancer is a peripheral decarboxylase inhibitor (e.g., carbidopa or benserazide).

In some embodiments, the dosage form comprises at least a first polymeric matrix material. In some embodiments, the first polymeric matrix material can swell in aqueous solutions (e.g., water and/or gastric fluid), thereby providing a swellable dosage form that increases in size to promote retention of the dosage from in the stomach. In some embodiments, the dosage form swells in the presence of gastric fluid to at least about 150% of a pre-swelling volume of the dosage form. In some embodiments, the dosage form swells in the presence of gastric fluid to at least about 200% of a pre-swelling volume of the dosage form. In some embodiments, the dosage form swells in the presence of gastric fluid to at least about 250% of a pre-swelling volume of the dosage form. In some embodiments, the dosage form swells in the presence of gastric fluid to at least about 300% of a pre-swelling volume of the dosage form. In some embodiments, the first polymeric matrix material comprises a hydrophilic polymer. In some embodiments, the hydrophilic polymer is selected from polyoxyethylene oxide, hydroxyethylcellulose, carboxymethylcellulose, polyethylene glycol diacrylate (PEGDA), gelatin, gelatin-PEGDA copolymer, hyaluronic acid, chitosan, hydroxypropylcellulose, hydroxypropylmethylcellulose, sodium acrylate, and copolymers thereof. In some embodiments, the first hydrophilic polymeric matrix material is crosslinked. In some embodiments, the first hydrophilic polymeric matrix material is non-crosslinked.

In some embodiments, the 5-HTP or pharmaceutically acceptable salt or solvate thereof is directly dispersed in the first polymeric matrix material. In some embodiments, the 5-HTP or pharmaceutically acceptable salt or solvate thereof is directly dispersed in the first polymeric matrix material in an amount between about 1 weight % (wt %) and about 50 wt % based on the weight of the first polymeric matrix material.

In some embodiments, the dosage form further comprises a plurality of microparticles dispersed within the first polymeric matrix material. In some embodiments, each of the plurality of microparticles comprises a second polymeric matrix material (e.g., comprising a hydrophilic polymer that can be the same as or different than a hydrophilic polymer of the first matrix material). In some embodiments, each of the microparticles further comprises 5-HTP or a pharmaceutically acceptable salt or solvate thereof. When the dosage form further comprises microparticles comprising 5-HTP or a pharmaceutically acceptable salt or solvate thereof, the amount of 5-HTP or pharmaceutically acceptable salt or solvate thereof directly dispersed in the first matrix material can be between about 0 wt % and about 50 wt % based on the weight of the first polymeric matrix material.

In some embodiments, the second polymeric matrix material comprises a crosslinked polymeric matrix material and/or a non-crosslinked polymeric matrix material. In some embodiments, the crosslinked polymeric matrix material comprises one or more hydrophilic polymer selected from the group comprising hydroxyl propyl methyl cellulose, hydroxyl propyl cellulose, hyaluronic acid, chitosan, gelatin, gelatin-PEGDA, PEGDA, and sodium acrylate. In some embodiments, the non-crosslinked polymeric matrix material comprises one or more hydrophilic polymer selected from the group comprising chitosan, poly(ethylene oxide), hydroxyl propyl cellulose and hydroxypropyl methylcellulose.

In some embodiments, the first polymeric matrix material contains between about 5 wt % and about 50 wt % of the microparticles (i.e., compared to the weight of the first polymeric matrix material). In some embodiments, each microparticle comprises between about 1 wt % and about 30 wt % of 5-HTP or a pharmaceutically acceptable salt or solvate thereof based on the weight of the microparticle.

In some embodiments, the dosage form comprises between about 50 mg and about 1,800 mg of 5-HTP or a pharmaceutically acceptable salt or solvent thereof. In some embodiments, at least about 30 wt % of the 5-HTP or pharmaceutically acceptable salt or solvate thereof is released within about 4 hours of oral administration. In some embodiments, at least about 50 wt % of the 5-HTP or pharmaceutically acceptable salt or solvate thereof is released within about 4 to about 9 hours of oral administration.

In some embodiments, the dosage form further comprises an additional active agent. In some embodiments, the additional active agent is one or more of the group comprising a serotonin enhancing compound, a peripheral decarboxylase inhibitor (e.g., carbidopa or benserazide), and a gas swelling agent. In some embodiments, the additional active agent (e.g., a peripheral decarboxylase inhibitor) is present in one or more microparticles dispersed within the first polymeric matrix material (either alone or together with 5-HTP).

In some embodiments, the dosage form is adapted to deliver a release profile of 5-HTP of between about 1 mg/hr and about 42 mg/hr of 5-HTP for a period of about 12 hours. In some embodiments, the release profile is substantially linear. In some embodiments, the dosage form provides a release rate (e.g., having a linear release profile) to the upper gastrointestinal tract of about 6.25 mg/hr, so as to provide an average steady state 5-HTP plasma level of about 0.25 mg/L.

In a third aspect of the invention, there is provided a method of treating a condition selected from the group comprising depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, the method comprising administering a gastroretentive and SR dosage form of 5-HTP as described in the first and/or second aspect of the invention to a patient in need thereof. In some embodiments, the dosage form is administered once or twice daily. In some embodiments, the dosage form is administered with a meal. In some embodiments, the dosage form is administered once or twice daily and the total amount of 5-HTP in the daily dosage is between about 50 mg and about 3,600 mg.

In some embodiments, the dosage form is adapted to deliver a release profile (e.g., a linear release profile) of between about 4 mg/hr and about 42 mg/hr of 5-HTP (e.g., to the upper GI) for a period of about 12 hours. In some embodiments, administration of the dosage form achieves a steady state 5-HTP plasma level of between about 0.1 mg/L and about 0.9 mg/L. In some embodiments, the method further comprises concomitant administration of a 5-HTP absorption enhancer to increase the steady state 5-HTP between about 1-fold and about 4-fold as compared to when the 5-HTP is administered without the absorption enhancer. In some embodiments, the 5-HTP absorption enhancer is a peripheral decarboxylase inhibitor, e.g., carbidopa or benserazide.

In a fourth aspect of the invention, there is provided a dosage form as described in the first and/or second aspect of the invention for use in the treatment of a condition selected from the group comprising depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment in a patient in need of such treatment.

In a fifth aspect of the invention, there is provided a use of a dosage form as described in the first and/or second aspect of the invention in the preparation of a medicament for the treatment of a condition selected from the group comprising depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment in a patient in need of such treatment.

According to a sixth aspect of the invention, there is provided a method of achieving steady state 5-HTP plasma levels of between about 0.1 mg/L and about 4 mg/L by administering 5-HTP or a pharmaceutically acceptable salt or solvate thereof to the upper GI at a release rate of between about 2.5 mg/hr and about 75 mg/hr. In some embodiments, the steady state 5-HTP plasma level is between about 0.1 mg/L and about 3 mg/L.

According to a seventh aspect of the invention, there is provided a method of achieving 5-HTP steady state plasma levels of between about 0.1 mg/L and about 1 mg/L by administering 5-HTP or a pharmaceutically acceptable salt or solvate thereof to the upper GI at a release rate of between about 2.5 mg/hr and about 25 mg/hr. In some embodiments, the method achieves a steady state 5-HTP plasma level of about 0.25 mg/L by administering 5-HTP or a pharmaceutically acceptable salt or solvate thereof to the upper GI at a release rate of about 6.25 mg/hr.

According to an eighth aspect of the invention, there is provided a method of achieving 5-HTP steady state plasma levels of between about 0.2 mg/L and about 2 mg/L by administering 5-HTP to the upper GI tract together with a peripheral decarboxylase inhibitor that enhances 5-HTP bioavailability by about 1-fold (to F=40%) at a release rate of 5-HTP of between about 2.5 mg/hr and about 25 mg/hr.

According to a ninth aspect of the invention there is provided a method of achieving 5-HTP steady state plasma levels of between about 0.3 mg/L and about 3 mg/L by administering 5-HTP to the upper GI tract together with an peripheral decarboxylase inhibitor that enhances 5-HTP bioavailability by about 2-fold (to F=60%) at a release rate of 5-HTP of between about 2.5 mg/hr and about 25 mg/hr.

According to a tenth aspect of the invention, there is provided a method of achieving 5-HTP steady state plasma levels of between about 0.4 mg/L and about 4 mg/L by administering 5-HTP to the upper GI tract together with an peripheral decarboxylase inhibitor that enhances 5-HTP bioavailability by about 3-fold (to F=80%) at a release rate of 5-HTP of between about 2.5 mg/hr and about 25 mg/hr.

According to an eleventh aspect of the present invention there is provided a gastroretentive SR pharmaceutical composition (e.g., dosage form) for delivering 5-HTP and optionally other active ingredients (e.g., a serotonin enhancing compound and/or a peripheral decarboxylase inhibitor) to the upper GI tract, said composition comprising.

(a) a first hydrophilic, swellable polymeric matrix material; and

(b) 5-HTP or a pharmaceutically acceptable salt or solvate thereof and optionally other active ingredients directly dispersed within the first polymeric matrix material, wherein the 5-HTP (or pharmaceutically acceptable salt or solvate thereof) is in an amount of between about 1 wt % and about 50 wt % based on the weight of the first polymeric matrix material.

According to a twelfth aspect of the present invention there is provided a gastroretentive SR pharmaceutical composition for delivering 5-HTP and optionally other active ingredients to the upper gastrointestinal tract, comprising:

(a) a first hydrophilic, swellable polymeric matrix material;

(b) 5-HTP or a pharmaceutically acceptable salt or solvate thereof and optionally other active ingredients (e.g., a serotonin enhancing compound and/or a peripheral decarboxylase inhibitor) directly dispersed within the first polymeric matrix material, wherein the 5-HTP (or pharmaceutically active salt or solvate thereof) is in an amount of between about 0 wt % and about 50 wt % (e.g. between about 1 wt % and about 50 wt %) based on the weight of the first polymeric matrix material; and

(c) a plurality of microparticles dispersed within said first polymeric matrix material, each of said microparticles comprising a second polymeric matrix material and an amount of 5-HTP or a pharmaceutically acceptable salt or solvate thereof and optionally other active ingredients (e.g., serotonin enhancing compounds and/or peripheral decarboxylase inhibitors) dispersed within the second polymeric matrix material, wherein:

the first and second polymeric matrix materials both comprise swellable and crosslinked polymeric matrix materials.

According to a thirteenth aspect of the present invention there is provided a gastroretentive SR pharmaceutical composition for delivering 5-HTP and optionally other active ingredients to the upper gastrointestinal tract, comprising:

(a) a first hydrophilic, swellable polymeric matrix material;

(b) 5-HTP or a pharmaceutically acceptable salt or solvate thereof and optionally other active ingredients (e.g., a serotonin enhancing compound and/or peripheral decarboxylase inhibitor) directly dispersed within the first polymeric matrix material, wherein the 5-HTP (or pharmaceutically acceptable salt or solvate thereof) is in an amount of between about 0 wt % and about 50 wt % (e.g. between about 1 wt % and about 50 wt %) based on the weight of the first polymeric matrix material; and

(c) a plurality of microparticles dispersed within said first polymeric matrix material, each of said microparticles comprising a second polymeric matrix material and an amount of 5-HTP or a pharmaceutically acceptable salt or solvate thereof and optionally other active ingredients (e.g., a serotonin enhancing compound and/or a peripheral decarboxylase inhibitor) dispersed within the second polymeric matrix material, wherein:

the first and second polymeric matrix materials are both swellable polymeric matrix materials without cross-linking.

According to a fourteenth aspect of the present invention there is provided a gastroretentive SR pharmaceutical composition for delivering 5-HTP and optionally other active ingredients to the upper gastrointestinal tract, comprising:

(a) a first polymeric matrix material;

(b) 5-HTP or a pharmaceutically acceptable salt or solvate thereof and optionally other active ingredients (e.g., a serotonin enhancing compound and/or a peripheral decarboxylase inhibitor) directly dispersed within the first polymeric matrix material, wherein the 5-HTP (or pharmaceutically acceptable salt or solvate thereof) is in an amount of between about 0 wt % and about 50 wt % (e.g. between about 1 wt % and about 50 wt %) based on the weight of the first polymeric matrix material; and

(c) a plurality of microparticles dispersed within said first polymeric matrix material, each of said microparticles comprising a second polymeric matrix material and an amount of 5-HTP or a pharmaceutically acceptable salt or solvent thereof and optionally other active ingredients (e.g., a serotonin enhancing compound and/or a peripheral decarboxylase inhibitor) dispersed within the second polymeric matrix material, wherein:

the first polymeric matrix material is swellable and the second polymeric matrix material releases the 5-HTP, or pharmaceutically acceptable salt or solvate thereof, and any other optional active ingredients via diffusion into the first polymeric matrix.

According to a fifteenth aspect of the present invention there is provided a gastroretentive SR pharmaceutical composition for delivering 5-HTP and optionally other active ingredients to the upper gastrointestinal tract, comprising:

(a) a first polymeric matrix material;

(b) 5-HTP or a pharmaceutically acceptable salt or solvate thereof and optionally other active ingredients (e.g., a serotonin enhancing compound and/or a peripheral decarboxylase inhibitor) directly dispersed within the first polymeric matrix material, wherein the 5-HTP (or pharmaceutically acceptable salt or solvate thereof) is in an amount of between about 0 wt % and about 50 wt % (e.g. between about 1 wt % and about 50 wt %) based on the weight of the first polymeric matrix material; and

(c) a plurality of microparticles disposed within said first polymeric matrix material, each of said microparticles comprising a second polymeric matrix material and an amount of 5-HTP or pharmaceutically acceptable salt or solvate thereof and optionally other active ingredients (e.g., a serotonin enhancing compound and/or peripheral decarboxylase inhibitor) dispersed within the second polymeric matrix material, wherein:

the first polymeric matrix material is swellable and the second polymeric matrix material releases the 5-HTP or pharmaceutically acceptable salt or solvate thereof and any other optional active ingredients mainly via erosion into the first polymeric matrix.

According to a sixteenth aspect of the present invention there is provided a gastroretentive SR pharmaceutical composition for delivering 5-HTP and optionally other active ingredients to the upper gastrointestinal tract, comprising:

(a) a first polymeric matrix material;

(b) 5-HTP or a pharmaceutically acceptable salt or solvate thereof and optionally other active ingredients (e.g., a serotonin enhancing compound and/or a peripheral decarboxylase inhibitor) directly dispersed within the first polymeric matrix material, wherein the 5-HTP or pharmaceutically acceptable salt or solvate thereof is in an amount of between about 0 wt % and about 50 wt % (e.g. between about 1 wt % and about 50 wt %) based on the weight of the first polymeric matrix material; and

(c) a plurality of microparticles disposed within said first polymeric matrix material, each of said microparticles comprising a second polymeric matrix material and an amount of 5-HTP or a pharmaceutically acceptable salt or solvate thereof and optionally other active ingredients (e.g., a serotonin enhancing compound and/or peripheral decarboxylase inhibitor) dispersed within the second polymeric matrix material, wherein:

the first polymeric matrix material is swellable and the second polymeric matrix material embodied as microparticles substantially remains dispersed within the first polymeric matrix during gastroretentive drug delivery.

Pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, l-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulfuric acids; from organic acids, such as tartaric, acetic, citric, malic, lactic, fumaric, benzoic, glycolic, gluconic, succinic, arylsulfonic acids; and from metals such as sodium, magnesium, or preferably, potassium and calcium.

As mentioned above, also encompassed are any solvates of 5-HTP and the other compounds mentioned hereinbelow (e.g. used in combination therapies) and their salts. Preferred solvates are solvates formed by the incorporation into the solid state structure (e.g. crystal structure) of the compounds mentioned herein of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallizing the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Solvates can be hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc. of West Lafayette, Ind., USA, 1999, ISBN 0-967-06710-3.

While not explicitly mentioned hereinabove, it will be appreciated that the active pharmaceutical ingredients will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A SR tablet may in addition contain from 0 to 90% (w/w) of a release-controlling polymer (e.g. a swellable polymer). A SR tablet may in addition also contain from 0 to 90% (w/w) or more of a release-controlling polymer (e.g. a swellable polymer) or mix of different polymers. A controlled release tablet may in addition also contain from 0 to 90% (w/w) of a release-controlling matrix in the form of microparticulates.

In addition, the formulations mentioned herein may also contain a serotonin-enhancing compound and/or excess fumaric or maleic acid or another aforementioned acid used for salts, or, to enhance 5-HTP bioavailability, a peripheral decarboxylase inhibitor and salts and solvates thereof. It will be appreciated that the salts and solvates here are as defined hereinbefore.

Serotonin-enhancing compounds (and salts and solvates thereof) suitable for use include selective serotonin reuptake inhibitors (SSRIs), serotonin-norepinephrine reuptake inhibitors (SNRIs), tricyclic antidepressants (TCAs), atypical antidepressants, and monoamine oxidase inhibitors (MAOIs). Examples of serotonin-enhancing compounds that may be mentioned in embodiments of the invention include, but are not limited to, citalopram, escitalopram, fluoxetine, fluvoxamine, paroxetine, sertraline, venlafaxine, duloxetine, vilazodone, vortioxetine, moclobemide, tranylcypromine, trazodone, nefazodone, mianserin, mirtazapine, and phenelzine.

Peripheral decarboxylase inhibitors (and salts and solvates thereof) that may be mentioned herein include, but are not limited to, carbidopa, benserazide (Ro-4-4602), difluromethyldopa, and α-methyldopa. If, for example, carbidopa is used in combination with 5-HTP, the breakdown of 5-HTP by aromatic-L-amino-acid decarboxylase (DOPA decarboxylase or DDC) in the periphery is inhibited, and simultaneously the oral bioavailability of 5-HTP is increased. Aromatic-L-amino-acid decarboxylase is a high-capacity enzyme normally functioning far below saturation (Jacobsen, Jacob P R, el al. “Adjunctive 5-Hydroxytryptophan slow-release for treatment-resistant depression: clinical and preclinical rationale.” Trends in pharmacological sciences (2016), 37(11): 933-944). At lower oral doses, a peripheral decarboxylase inhibitor may therefore only reach pharmacologically active concentrations locally in the intestine and will thus mainly or only enhance 5-HTP bioavailability, with modest, minor, or no effects on 5-HTP metabolism by aromatic-L-amino-acid decarboxylase in the systemic circulation and non-gastric peripheral organs. When administered orally simultaneously with 5-HTP, a peripheral decarboxylase acts as a 5-HTP absorption enhancer, i.e. enhances 5-HTP's bioavailability. For a given unit dose of 5-HTP, it is known in the art that a peripheral decarboxylase can enhance 5-HTP bioavailability (Gijsman H J, van Gerven J M, de Kam M L, Schoemaker R C, Pieters M S, Weemaes M, de Rijk R, van der Post J, Cohen A F. “Placebo-controlled comparison of three dose-regimens of 5-hydroxytryptophan challenge test in healthy volunteers.” J Clin Psychopharmacol. (2002), 22(2):183-9. PubMed PMID: 11910264; Westenberg H G, Gerritsen T W, Meijer B A, van Praag H M. “Kinetics of l-5-hydroxytryptophan in healthy subjects.” Psychiatry Res. (1982), 7(3):373-85. PubMed PMID: 6187038). Co-administration of 5-HTP and a peripheral decarboxylase inhibitor can increase plasma 5-HTP levels (e.g. from one-fold to about fifteen-fold in some cases), meaning that the amount of 5-HTP needed in the dosage form may be reduced. Typically, co-administration with carbidopa at high doses that are systemically active doubles 5-HTP's half-life from about 2-hours to about 4-hours. At lower doses, peripheral decarboxylase inhibitors may mainly enhance bioavailability of 5-HTP, while at higher doses, e.g. at about 150 mg/day (Gijsman H J, van Gerven J M, de Kam M L, Schoemaker R C, Pieters M S, Weemaes M, de Rijk R, van der Post J, Cohen A F. “Placebo-controlled comparison of three dose-regimens of 5-hydroxytryptophan challenge test in healthy volunteers.” J Clin Psychopharmacol. (2002), 22(2):183-9. PubMed PMID: 11910264), both 5-HTP's bioavailability and half-life will be enhanced. 5-HTP easily crosses the blood-brain barrier. Therefore, an elevation of blood plasma 5-HTP levels can result in an increase in 5-HTP in the central nervous system (CNS) available for serotonin synthesis in the CNS. Indeed, elevated levels of plasma 5-HTP is known from animal and human studies to result in increased CNS serotonin levels (see Jacobsen et al., Neuropsychopharmacology (2016) 41:2324-2334). Peripheral decarboxylase inhibitors do not cross the blood-brain barrier. Therefore, co-administering a peripheral decarboxylase inhibitor to 5-HTP inhibits conversion of 5-HTP to serotonin in the periphery, allowing more 5-HTP to enter the CNS, resulting in an increase in synthesis and levels of serotonin in the CNS.

When used as part of the dosage form, the serotonin-enhancing compounds and peripheral decarboxylase inhibitors may be simply incorporated into the same compartments of the dosage form as 5-hydroxytryptophan, or they may be distributed in a different manner in the dosage form. For instance, the serotonin-enhancing and peripheral decarboxylase inhibitor compounds may be incorporated into the same matrix, separate compartments, separate matrices, separate layers or granules, the microparticles, coated particles, the coating, and/or incorporated as loose powder, particles, or solid sub-dosage forms into the capsule.

Compartments can be distinct oral sub-dosage forms bound together by a joining layer. Compartments can also be microparticles vs matrix, microparticles with different active pharmaceutical ingredients, and so forth. The sub-dosage forms can for instance comprise different active pharmaceutical compounds, and/or provide different release rates which combine to provide the overall release rate of the dosage form. Compartments can also be distinct microparticles, for instance encompassing different active pharmaceutical compounds, and/or providing different release rates which combine to provide the overall release rate of the dosage form. Different compartments can have different compositions of polymers and other excipients to accommodate different active pharmaceutical compounds to provide similar release rates for different compounds, and/or provide different release rates for the same compounds.

The amounts of the above compounds incorporated into the dosage form may be selected based on the ranges used in standard clinical practice or could be higher or lower as therapeutically required. Further, these compounds can be administered together with the 5-HTP gastroretentive SR formulation dosage form as separate dosage forms, including, but not solely, as a kit. Serotonin-enhancing compounds and peripheral decarboxylase inhibitors may be incorporated or used with the 5-HTP gastroretentive formulation dosage form as just described either individually or together. For example, the 5-HTP gastroretentive formulation dosage could co-incorporate one or more serotonin-enhancing compounds, one or more peripheral decarboxylase inhibitors, of both types of compounds together with 5-HTP, either integral with the 5-HTP gastroretentive formulation dosage form, separately, or as a kit.

As noted above, the dosage forms mentioned herein in relation to the first to sixteenth aspects of the invention comprise 5-HTP or pharmaceutically acceptable salts or solvates thereof. Unless otherwise mentioned herein, the weight of 5-HTP is the weight of the free base of 5-HTP. Any salts or solvates that may be used will accordingly have a higher mass value. The 5-HTP may conveniently be present in an amount of from about 50 mg to about 1000 mg, such as from about 50 mg to about 150 mg, such as from about 200 mg to 400 mg, such as from about 300 mg to about 700 mg, such as from about 400 to about 500 mg, or such as from about 700 to about 1,000 mg, in embodiments of the invention as described hereinbelow. In addition, to pharmaceutically acceptable salts and solvates, in some embodiments, an analog of 5-HTP can be included, in place of or in addition to the 5-HTP or pharmaceutically acceptable salt or solvate thereof. For example, in some embodiments, the analog is deuterated 5-HTP. In some embodiments, the analog is a pro-drug of 5-HTP.

As noted above, the dosage formulations disclosed herein, such as in the first to sixteenth aspects of the invention (and their embodiments) disclosed herein, act to release the 5-HTP (or pharmaceutically active salt or solvate thereof) over an extended period of time. For example, the formulations display a release profile in which:

at least about 30 wt % of the 5-HTP or pharmaceutically acceptable salt or solvate thereof is released within between about 3 hours and about 5 hours (e.g. within about 4 hours) of oral administration, and

up to about 100 wt % of the 5-HTP or pharmaceutically acceptable salt or solvate thereof is released within between about 8 hours and about 12 hours of oral administration. In some embodiments, at least about 80 wt % of the 5-HTP or pharmaceutically acceptable salt or solvate thereof is release within between about 6 hours and about 10 hours of oral administration (e.g., within about 6, 7, 8, 9, or 10 hours of oral administration).

In particular embodiments of the invention, such as but not limited to, in relation to the first and third to sixteenth aspects of the invention, the dosage form may be adapted to deliver a release profile of from between about 1 mg/hr and about 42 mg/hr of 5-HTP for a period of about 12 hours. For example, the dosage form may be adapted to deliver a release profile of from between about 4 mg/hr and about 42 mg/hr of 5-HTP for a period of 12 hours. In certain embodiments, the release profile of 5-HTP may be substantially linear. For the avoidance of doubt, reference to “substantially linear” herein may refer to both in vivo and, more particularly, to in vitro release profile tests and/or measurements.

There are a number of possible dosage forms that may provide the desired release profile mentioned above, these forms will be discussed in more detail below.

### III.A. Swellable Systems

The swellable dosage form systems disclosed herein use generally regarded as safe (GRAS) materials or excipients on the FDA's Inactive Ingredient List.

In one some embodiments, the dosage form may comprise at least one polymeric matrix material that comprise a hydrophilic polymer that swells to an extent that it promotes gastric retention of the dosage form of the gastroretentive SR dosage form following administration to a subject, e.g., in the fed state. The 5-HTP (or salt or solvent thereof) may be presented as a single particle (i.e. a monolithic particle of 5-HTP) or, more particularly, as a plurality of solid particles (e.g. in combination with suitable excipients and the like) dispersed in the polymeric matrix material.

For example, the hydrophilic polymer may swell in contact with gastric fluid to such an extent that the dosage form is too large to pass through the pyloric sphincter, thereby retaining the tablet in the stomach for an extended period of time, such as up to about 12 hours. Over this period, 5-HTP is slowly released through diffusion and/or erosion of the polymer and thus 5-HTP, and any co-incorporated active ingredient(s), e.g. a peripheral decarboxylase inhibitor and/or a serotonin enhancing compound, is gradually released to the stomach, duodenum and small intestine of the patient (i.e. the upper GI-tract). When a peripheral decarboxylase inhibitor, a serotonin enhancing compound, or other active ingredient is included in the dosage form, it may be in the same polymeric matrix material as the 5-HTP (or salt or solvate thereof) or in a different polymeric matrix. When there are two polymeric matrices, the two matrices may be in the same layer of a tablet or in different layers. The decarboxylase inhibitor, serotonin enhancing compound, or other active ingredient may be included in the matrix/matrices, in a coating, as a coated particle, granule, pellet, or bead, or as uncoated particles, granules, and so on.

As an example of such a system, the gastroretentive SR dosage form may contain a polymer(s) with a high swelling capacity such as, but not limited to, one or more of polyethylene oxide, hydroxyethylcellulose, carboxymethylcellulose, and hydroxypropylmethylcellulose (e.g. the polymeric matrix may comprise of poly(ethylene oxide) and hydroxypropylmethylcellulose). The polymers that form the polymeric matrix may have a moderate to high molecular weight (e.g. the polymers may have a weight average molecular weight of at least about 5×10⁴ Daltons, such as from 5×10⁴ to 1×10⁷ Daltons) to enhance swelling and provide control of the release of the 5-HTP.

The gastroretentive SR dosage form may contain a dose of a peripheral decarboxylase inhibitor that enhances the bioavailability of 5-HTP by 1-fold to 4-fold (from F=20% to F=100%). The dose of peripheral decarboxylase inhibitor may also be thus adjusted to enhance the half-life of 5-HTP, e.g. to approximately 2 h, 2.5 h, 3 h, 3.5 h, and 4 h.

In some embodiments, the swellable system (e.g., the dosage form) can also include one or more microparticles dispersed within the swellable polymer or polymers (i.e., the first polymeric matrix material). Examples of microparticles dispersed in the first polymeric matrix material include, but are not limited to, microbeads, crystals, nanoparticles, minitablets, beads, pellets, and granulates. The 5-HTP (or salt or solvent thereof) can be dispersed within the microparticles which are dispersed in the first matrix material (i.e., the 5-HTP can be indirectly dispersed in the first matrix material), or directly dispersed in the first matrix material, or both.

The above-mentioned system may be prepared using common techniques available to a person skilled in the art (see e.g., U.S. Pat. Nos. 6,340,475; 6,635,280; 7,438,927; and 9,161,911).

Thus, in a further swellable system embodiment, the dosage form may comprise:

(a) a first polymeric matrix material;

(b) 5-HTP (or a pharmaceutically acceptable salt or solvate thereof) directly dispersed within the first polymeric matrix material in an amount between about 0 wt % and about 50 wt % (e.g. between about 1 wt % and about 50 wt %) based on the weight of the first polymeric matrix material; and

(c) a plurality of microparticles disposed within said first polymeric matrix material, each of said microparticles comprising a second polymeric matrix material and an amount of 5-HTP, or a pharmaceutically acceptable salt or solvate thereof, dispersed within the second polymeric matrix material, wherein:

the first polymeric matrix material is a swellable non-crosslinked polymeric matrix material.

In a further swellable system, the dosage form may comprise:

(a) a first polymeric matrix material;

(b) 5-HTP (or a pharmaceutically acceptable salt or solvate thereof) directly dispersed within the first polymeric matrix material in an amount of between about 0 wt % and about 50 wt % (e.g. between about 1 wt % and about 50 wt %) based on the weight of the first polymeric matrix material; and

(c) a plurality of microparticles disposed within said first polymeric matrix material, each of said microparticles comprising a second polymeric matrix material and an amount of 5-HTP, or a pharmaceutically acceptable salt or solvate thereof, dispersed within the second polymeric matrix material, wherein:

the first polymeric matrix material is a swellable and crosslinked polymeric matrix material

When used herein, the terms “swellable” and “swells” may be interpreted with regard to the discussion of the term “swells” hereinbefore. That is, the dosage form may swell to approximately 115% to 150% or greater of its dry original volume within one hour after administration (or being placed in an aqueous vessel), and at a later time may swell to a volume that approximately from 130% to 300% or greater of its original dry volume. Alternatively, “swellable” may refer to the ability of a polymeric matrix to absorb an amount of water (or gastric fluid), for example the polymeric matrix may be capable of swelling in water or gastric fluid to a weight in the range of 1.5 to 10 times its weight in a dehydrated form over time. The rate of swelling should be less than 50% in the first 5 to 10 minutes to avoid problems with swallowing or choking. Swelling can be measured in a USP dissolution vessel by removing the tablet at fixed times and measuring weight, volume, or density.

When used herein, “directly dispersed within the first polymeric matrix material” refers to particles of an active ingredient (e.g. 5-HTP), which may be presented as a free base, salt, or solvate and which particles may optionally also contain a conventional pharmaceutically acceptable carrier material that is not a polymeric matrix material, in direct contact with the first polymeric matrix material. It will be appreciated that an active ingredient dispersed within a second polymeric matrix material is not directly dispersed within the first polymeric material. In certain embodiments, when 5-HTP is present in the first polymeric matrix material alone, it may be presented as particles of the free base or a salt or solvate. A peripheral decarboxylase inhibitor or a serotonin enhancing compound may optionally be included in a manner analogous to 5-HTP.

As mentioned above, fillers, binders, lubricants and other additives may also be included in the gastric retained dosage form, such as are well known to those of skill in the art.

The first polymeric material may be any suitable crosslinked swellable polymer and, as such, may be selected from one or more of the group that includes, but is not limited to, PEGDA, gelatin (e.g. gelatin+genipin), gelatin-PEGDA, crosslinked hyaluronic acid, crosslinked hydroxyl propyl cellulose, crosslinked hydroxyl propyl methyl cellulose and crosslinked sodium acrylate. For example, the first polymeric material may be gelatin-PEGDA. A further crosslinked material that may be mentioned herein is crosslinked chitosan (e.g. a chitosan with a degree of de-acetylation ranging from 20-50%, which has been crosslinked with a suitable crosslinking agent (e.g. epichlorhydrin or glutaraldehyde under coacervation conditions)).

Alternatively, the first polymeric material may be any suitable non-crossed linked swellable polymers with a high swelling capacity, such as polyethylene oxide, hydroxyethylcellulose, and hydroxypropylmethylcellulose or a combination of these. The polymers are preferably of a moderate to high molecular weight (about 5×10⁵ Daltons to greater than about 10⁷ Daltons) to enhance swelling, retention in the stomach, and to provide control of the release of the 5-HTP and other incorporated active compounds.

The second polymeric material, which forms the microparticles, may be made of a crosslinked polymeric material selected from one or more of the group including, but not limited to, hydroxyl propyl methyl cellulose, hydroxyl propyl cellulose, hyaluronic acid, chitosan, gelatin, gelatin-PEGDA, PEGDA, and polyacrylic acids (including their salts such as sodium acrylate), or it may be an non-crosslinked polymeric material selected from one or more of the group consisting of chitosan (e.g. a chitosan with a degree of de-acetylation ranging from 20-50%), poly(ethylene oxide), hydroxyl propyl cellulose and hydroxypropyl methylcellulose. It will be appreciated that these polymeric materials may also exhibit a degree of swelling in a liquid environment. In other words, the second polymeric matrix may exhibit a degree of swelling. Without wishing to be bound by theory, this swelling may help contribute to keeping the dosage form in the stomach.

Polymers described herein that display swelling may particularly swell when in a liquid environment that has a low pH value (i.e. a pH of less than 7), and several show pH independent swelling over the entire physiological pH range.

Crosslinked polymers mentioned herein may be crosslinked by any suitable method depending on the polymer in question, such as by chemical crosslinking (e.g. crosslinking through the use of a multi-valent cation (e.g. a cation with a 2+ or 3+ charge, such as Ca²⁺ and Fe³⁺) or by the use of a chemical crosslinking agent, such as genipin which may be used to crosslink gelatin) or by other methods of crosslinking, such as by ultra violet light crosslinking (e.g. where the polymer itself contains moieties that may crosslink together upon exposure to ultraviolet light). Any suitable degree of crosslinking may be used herein, which may be measured using crosslink density (Mc). Crosslink density is defined herein as the molar mass between crosslinks and may range from a few thousand Daltons to a few Daltons. Following crosslinking, any remaining free crosslinker, crosslinking initiator, or the like should be removed from the dosage form.

In some embodiments of the dosage form above, the 5-HTP, or pharmaceutically acceptable salt or solvate thereof, is dispersed within the second polymeric matrix material and these materials together form microparticles, which are disposed within the first polymeric matrix material (e.g. homogeneously dispersed within the first polymeric matrix). Any suitable loading of 5-HTP may be used in the microparticles. Suitable loading values that may be mentioned herein include embodiments where the 5-HTP, or pharmaceutically acceptable salt or solvate thereof, is present in an amount of between about 1 wt % and about 50 wt % (e.g. between about 1 wt % and about 30 wt %) of each microparticle. The first polymeric matrix material may contain between about 5 wt % and about 50 wt % (e.g. between about 10 wt % and about 45 wt %) of said microparticles.

While not wishing to be bound by theory, it is believed that in this dosage form following administration, the 5-HTP is released from the microparticles into the first polymeric matrix and then diffuses through the first polymeric matrix into the gastric fluid. It is possible that some fraction of the microparticles also efflux intact with the drug into the gastric fluid, and then release 5-HTP directly into the gastric fluid (or into a fluid lower down the GI-tract). In addition, when the composition contains 5-HTP in the first matrix, this 5-HTP will diffuse directly into the gastric fluid. In some embodiments, the main element of slow-release delivery of the active ingredient will be provided by the microparticles, in some embodiments the slow-release delivery will be provided substantially by both the microparticles and the first matrix.

It will be appreciated that the drug loading, the crosslink density of the microparticles, the size of microparticles and the concentration of microparticles within the first polymeric matrix may be varied to achieve a particular release profile. The nature of the composition will also influence the release rate.

The above-mentioned swellable system may be prepared using common techniques available to a person skilled in the art. For example, microparticles can be produced by a simple water-in-oil emulsion method, where the non-crosslinked polymer and excipients, including the active pharmaceutical ingredient, will be dissolved in water, and then emulsified into an organic solvent. The solvent is then evaporated off, and the residue may be UV-crosslinked and lyophilized to yield particles. Another way is to use multilamellar liposomes as a template. In this case liposomes are formed by drying a solution of lipids into a film form, then hydrating this with an aqueous solution consisting of the crosslinkable polymer, excipients and the active pharmaceutical ingredient. The resulting liposome may then be UV-crosslinked and dialyzed to remove uncrosslinked material. The lipid bilayer is then stripped off with detergent to yield gel particles. The microparticles may then be incorporated by mixing into a swellable matrix prepared from crosslinked polymers, in a manner known to a person skilled in the art. The composition so-produced can be used to fill capsules in a manner known to a person skilled in the art.

The swelling formulations discussed above may also contain, in certain embodiments a gas generating agent. When the swellable gastroretentive SR formulations described herein are brought into contact with gastric juice, the gas swelling agent generates a gas in at least part of the formulation, which may allow the formulation to float in the gastric juice of the stomach and intestines for a period of time. This may provide the formulations described in this section with an additional buoyancy soon after oral administration, which may help to avoid inadvertent passage of the dosage form through the pyloric sphincter before the swellable polymers described above have had sufficient time to swell to a size that cannot pass through the pyloric sphincter. Thus, the optional inclusion of a gas swelling agent may help to enhance gastric retention. Floating gastric retentive systems are described in U.S. Pat. Nos. 4,140,755; 4,996,058; and 6,960,356; and in Timmermans, Moes, A J, J. Pharm. Sci. (1994), 83:18-24.

The rapid release of the gas from the gas swelling agent may occur within a short period of time following oral administration (e.g. five minutes). Any suitable gas swelling agent (i.e. any suitable gas generating material) may be used, provided that it releases gas upon contact with gastric juices. Suitable gas swelling agents include, but are not limited to, uni- or bi-valent basic salts of carbonic acid (i.e. carbonates and bicarbonates) such as sodium hydrogen carbonate, sodium carbonate, sodium bicarbonate, potassium carbonate, potassium bicarbonate, calcium carbonate, magnesium carbonate and sodium glycine carbonate; and sulfites such as sodium sulfite, sodium bisulfite and sodium metabisulfite. Optionally acids such as citric acid, malic acid, maleic acid, fumaric acid, tartaric acid, and other aforementioned acids may be included to react with the gas generating agent when it is wetted.

The gas generating material may be employed in an amount ranging between about 0.1 wt % and about 50 wt %, such as between about 1 wt % and about 10 wt % based on the total weight of the dosage form.

The incorporation of a gas swelling agent into the dosage forms may use common techniques available to a pharmacist (e.g. as described in European patent application No. EP 2120887, which techniques are incorporated herein by reference).

It will be appreciated that the dosage forms discussed above may be provided in a form where they are contained within a capsule, typically a gelatin capsule, to facilitate swallowing.

### III.B. Other Systems

In some embodiments, the gastroretentive SR dosage form may employ Intec Pharma's ‘Accordion’ technology. In such embodiments, 5-HTP and, optionally, other active ingredients, are incorporated into a biodegradable polymeric film. The film is a multi-layer, planar structure, folded to an accordion shape and packed into a standard size capsule. Upon reaching the stomach, the capsule dissolves. The film then unfolds and is of substantial size, so is retained in the stomach for up to 12 hours. While in the stomach, the film releases the drug in a controlled manner to the upper part of the gastrointestinal tract. This dosage form may be particularly suited to a combination dosage form (e.g. comprising 5-HTP and a serotonin-enhancing compound, a peripheral decarboxylase inhibitor, or all three compounds (e.g. (i) 5-HTP and carbidopa; (ii) 5-HTP and a selective serotonin re-uptake inhibitor; or (iii) 5-HTP, a selective serotonin re-uptake inhibitor, and carbidopa)). The formulations described in U.S. Pat. No. 8,771,730, which relate to L-Dopa (e.g. in combination with carbidopa) are hereby incorporated by reference (with L-Dopa being replaced by 5-HTP).

In some embodiments, the gastroretentive SR dosage form may employ Lyndra Therapeutic's technology. In such embodiments, 5-HTP and, optionally, other active ingredients, such as a peripheral decarboxylase inhibitor or a serotonin enhancing compound, are incorporated into carrier polymer-ingredient components comprising i) a carrier polymer, and ii) a therapeutic ingredient or a pharmaceutically-acceptable salt thereof, wherein the carrier polymer-agent components are linked together by one or more coupling polymer components, wherein at least one of the one or more coupling polymer components is an elastomer; wherein the gastric residence systems are configured to have a compacted form in a container, suitable for administration orally or through a feeding tube; and an uncompacted form, such as a ring or a star, when released from the container; wherein the gastric residence systems are retained in the stomach for a residence period of 8 h to 24 h, or longer. See U.S. Patent Application Publication Nos. 2017/0266112; and 2018/0311154.

Also disclosed herein, the sustained release of 5-HTP may be achieved by way of a subcutaneous or intramuscular injectable slow-release formulation of 5-HTP.

### III.C. Sizes and Shapes of Dosage Forms

Generally, the tablet or capsules of the presently disclosed dosage forms will have a long axis and a short axis. This shape feature will 1) facilitate ingestion and passage through the mouth and oesophagus and 2) assist in retaining the dosage form in the stomach after swelling. Such benefits are described in U.S. Pat. No. 6,488,962, which is incorporated herein for reference. Upon swelling in the stomach, within 30-60 min, the shorter axis will swell to at least 1.2 cm, preferably 1.3 cm or more, which is a size too large to pass through the mean size pyloric sphincter in the fed state. Upon swelling in the stomach, the longer axis will swell to at least 2 cm, preferably 2.5 cm or more, and most preferably 3 cm or more. Before swelling, the shorter axis can be as short as 0.7 cm, preferably 0.7 cm to 1.5 cm in length, and preferably 0.75 cm to 1.2 cm in length, and most preferably 0.8 cm to 1.0 cm in length. The longer axis of the tablet prior to swelling will be 3.0 cm or less in length, preferably 2.5 cm or less, and most preferably 1.5 cm to 2.5 cm.

Preferable shapes include, but are not limited to, shapes that are oblong, diamond shaped, oval, cylindrical, and parallelogramic. The thickness of the tablet will be equal to or less than the dimensions of both the long and short axis.

### III.D. Dosage and Treatment

5-HTP may be administered in a therapeutically effective amount for the treatment of a CNS disorder, such as a condition selected from the group including, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment. Typically, the method of the invention will involve administering the gastroretentive SR 5-HTP dosage form on a once- or twice-daily basis for as long as the condition persists. In some embodiments it can on a thrice-daily basis.

A peripheral decarboxylase inhibitor can be incorporated in the dosage form to enhance 5-HTP's bioavailability or to enhance both 5-HTP's bioavailability and plasma elimination half-life simultaneously.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states to the extent that of these are possible.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult, juvenile, and newborn subjects, whether male or female, or not identifying as any specific gender, are intended to be covered.

As used herein the term “therapeutically effective amount” refers to that amount which is sufficient to effect treatment, when administered to a mammal in need of such treatment (e.g. sufficient to treat or prevent the disease). The effect may be objective (i.e. measurable by some test or marker) or subjective (i.e. the subject gives an indication of or feels an effect). The therapeutically effective amount will vary depending on the subject being treated, the severity of the disease state and the manner of administration, and may be determined routinely by the person skilled in the art.

An effective dosage of 5-HTP, whether alone, with a peripheral decarboxylase inhibitor, with a serotonin-enhancing compound, or with both, is typically in the range of about 50-3600 mg/day, typically about 300-2400 mg/day, more typically about 600-1800 mg/day.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, the age, condition, body weight, sex and response of the patient to be treated, and the stage/severity of the disease.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

In some embodiments, a once- or twice-daily dose of the gastroretentive SR 5-HTP dosage form is administered. The dosage can be administered at any time, but it is preferred that the dosage is administered at the same approximate time each day and at approximately 12 hour intervals for the duration of treatment. In addition, it is preferred that the gastroretentive SR 5-HTP dosage form be taken with food, for example with the morning or evening meals. Accordingly, in some embodiments, the gastroretentive SR 5-HTP dosage form is administered once-daily, for example, in the morning (e.g., upon rising or with the morning meal) or in the evening (e.g., with the evening meal or near bedtime). In some embodiments, the gastroretentive SR 5-HTP dosage form is administered twice-daily, for example, with the first dose being taken in the morning (e.g., upon rising or with the morning meal) and the second dose being in the evening (e.g., with the evening meal or near bedtime).

In some embodiments, the meal causes a cessation of the periodic intense bursts of peristaltic waves associated with the fasting mode, specifically the Phase III of the interdigestive migrating motor complex. The fed mode is induced by nutritive elements immediately after food ingestion and begins with a rapid and profound change in the motor pattern of the upper gastrointestinal (GI) tract. The change occurs almost simultaneously at all sites of the GI tract, before the stomach contents have reached the distal small intestine. During the fed mode, the stomach generates 3-4 continuous and regular contractions per minute, similar to those of the fasting mode but with about half the amplitude. The pyloric sphincter is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 6 hours, allowing for the dosage form to swell to a size sufficiently for prolonged retention and residence time, e.g. up to about 12 hours, or longer, in the stomach.

### III.E. Combination Therapy

In accordance with some embodiments, the gastroretentive SR 5-HTP dosage from may be administered alone (i.e. as a monotherapy, such as a monotherapy for the treatment of depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment). In some embodiments, however, the gastroretentive 5-HTP SR dosage form may be administered in combination with another therapeutic agent (e.g. another therapeutic agent for the treatment of depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment).

Therefore, pharmaceutical treatments according to the invention comprising 5-HTP and a pharmaceutically acceptable carrier may further comprise one or more additional therapeutic agents. Pharmaceutical compositions containing, in addition to 5-HTP, a serotonin-enhancing compound and/or a peripheral decarboxylase inhibitor have already been described hereinbefore. It will be appreciated that these components may be provided and administered to the subject separately.

Thus, further aspects of the invention relate to the following:

(a) a gastroretentive SR 5-HTP dosage form, as hereinbefore defined, and another therapeutic agent for use in the treatment of a CNS disease or disorder, such as, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, wherein the gastroretentive SR 5-HTP dosage form, as hereinbefore defined, may be administered sequentially, simultaneously or concomitantly with the other therapeutic agent;

(b) a gastroretentive SR 5-HTP dosage form, as hereinbefore defined, for use in the treatment of a CNS disease or disorder, such as, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, wherein the gastroretentive SR 5-HTP dosage form is administered sequentially, simultaneously or concomitantly with another therapeutic agent;

(c) Use of a gastroretentive SR 5-HTP dosage form, as hereinbefore defined and another therapeutic agent, for the preparation of a medicament for the treatment of a CNS disease or disorder, such as, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, wherein the gastroretentive SR 5-HTP dosage form is administered sequentially, simultaneously or concomitantly with the other therapeutic agent;

(d) Use of a gastroretentive SR 5-HTP dosage form, as hereinbefore defined, for the preparation of a medicament for the treatment of a CNS disease or disorder, such as, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, optionally wherein the medicament is administered in combination with another therapeutic agent;

(e) A method of treatment of a CNS disease or disorder, such as, but not limited to, depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment, which method comprises the administration of an effective amount of a gastroretentive SR 5-HTP dosage form, as hereinbefore defined, and another therapeutic agent to a patient in need of such treatment.

When used herein, the term “another therapeutic agent” includes references to one or more (e.g. one) therapeutic agents (e.g. one therapeutic agent) that are known to be useful for (e.g. that are known to be effective in) the treatment of CNS diseases or disorders, such as depression, social anxiety, panic disorder, generalized anxiety disorder, OCD, impulse control disorders, suicidality, borderline personality disorder, fibromyalgia, ataxia, mood symptoms and agitation related to neurological disorders (e.g. Alzheimer's disease, Parkinson's disease), stroke recovery, autism, migraine, sleep disorders, premenstrual dysphoria, post-traumatic stress disorder, post-partum depression, phenylketonuria, and depression after interferon treatment. In particular embodiments, these another therapeutic agents may be selected from one or more serotonin-enhancing compounds and/or peripheral decarboxylase inhibitors, which are as defined hereinbefore.

The dose of 5-HTP may be as defined hereinbefore, optionally modified to take into consideration the combination therapy. The dose of the another therapeutic agents may be determined by a medical practitioner in line with the considerations discussed hereinbefore for determination of the dose of 5-HTP when used alone.

The another therapeutic agents can be administered in whatever form they can be appropriately used for therapeutic purposes in humans. Different ‘therapeutic agents’ will typically need different dosage forms. However, for example, the peripheral decarboxylase inhibitor, carbidopa may work well when released at a similar rate as the 5-HTP as it has somewhat similar physiochemical properties and would be expected to behave more or less similarly to 5-HTP in most formulations. As such, when carbidopa is used in an embodiment of the invention it may be conveniently incorporated into the 5-HTP gastroretentive SR formulation.

When used herein, the term “administered sequentially, simultaneously or concomitantly” includes references to: administration of separate pharmaceutical formulations (one containing the gastroretentive SR 5-HTP dosage form and one or more others containing the one or more other therapeutic agents); and administration of a single pharmaceutical formulation containing the gastroretentive SR 5-HTP dosage form and the other therapeutic agent(s).

The combination product described above provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)). Thus, there is further provided a kit of parts comprising components: (i) a pharmaceutical formulation including a gastroretentive SR 5-HTP dosage form, as hereinbefore defined; and (ii) a pharmaceutical formulation including another therapeutic agent, in admixture with a pharmaceutically-acceptable adjuvant, diluent or carrier, wherein components (i) and (ii) are each provided in a form that is suitable for administration in conjunction with the other. Component (i) of the kit of parts is thus component (A), which is a formulation of 5-HTP as described hereinbefore. Similarly, component (ii) is component (B) in admixture with a pharmaceutically-acceptable adjuvant, diluent or carrier.

It will be appreciated that the above dosage forms (including the combinations) may provide a substantially linear release rate of 5-HTP into the upper GI tract of a subject. As such, there is provided a method of achieving 5-HTP plasma levels of from about 0.1 mg/L to 1 mg/L by administering from about 2.5 mg/hr to about 25 mg/hr at steady state to the upper gastrointestinal tract (e.g. the method may achieve steady state 5-HTP plasma levels of about 0.25 mg/L by administering about 6.25 mg/hr at steady state to the upper gastrointestinal tract). As such, there is provided a method of achieving 5-HTP plasma levels of from about 0.1 mg/L to 3 mg/L by administering from about 2.5 mg/hr to about 75 mg/hr at steady state to the upper gastrointestinal tract (e.g. the method may achieve steady state 5-HTP plasma levels of about 0.25 mg/L by administering about 6.25 mg/hr at steady state to the upper gastrointestinal tract). It will be appreciated that any of the dosage forms disclosed herein that meet the release criteria described in these aspects of the invention may be used. It will be appreciated that inclusion of a peripheral decarboxylase inhibitor in the dosage form can increase the steady state plasma levels of 5-HTP resulting from a given delivery rate (as described in the foregoing) by 1-fold to by 4-fold.

As mentioned hereinbefore, the invention may also relate to a specific dosage form that may comprise any suitable active ingredient that would benefit from a gastroretentive SR formulation. As such, there is provided a gastroretentive SR pharmaceutical composition for delivering an active pharmaceutical ingredient to the upper gastrointestinal tract, comprising:

(a) a first polymeric matrix material;

(b) a first active ingredient (e.g., 5-HTP) or a pharmaceutically acceptable salt or solvate thereof directly dispersed within the first polymeric matrix material in an amount of between about 0 wt % and about 50 wt % (e.g., between about 1 wt % and about 50 wt %) based on the weight of the first polymeric matrix material; and

(c) a plurality of microparticles disposed within said first polymeric matrix material, each said of said microparticles comprising a second polymeric matrix material and an amount of a second active ingredient, or a pharmaceutically acceptable salt or solvate thereof, dispersed within the second polymeric matrix material, wherein the first polymeric matrix material is swellable. Each of the first and second polymeric matrix materials can be crosslinked or non-crosslinked. In some embodiments, the second polymeric matrix material is swellable. In some embodiments, both the first and second polymeric matrix materials are swellable and crosslinked. In some embodiments, the first and second polymeric matrix materials are both swellable and non-crosslinked.

In embodiments of this aspect, the first and second polymeric matrix materials may be the same as discussed hereinbefore for the first to fourth aspects of the invention. The first and second active ingredients may be any active ingredient(s) that would benefit from a gastroretentive SR delivery approach. In certain embodiments, the first and second active ingredient may each be independently selected from the group comprising 5-HTP, carbidopa, benserazide, L-DOPA, garbapentin, metformin, amoxicillin, metronidazole, clarithromycin, nitrofurantoin, acyclovir, furosemide, captopril, metoprolol, ranitidine, famotidine, ciprofloxacin, ofloxacin, verapamil, atenolol, baclofen, ciprofloxacin, cefuroxime axetil, celecoxib, diltiazem, metoclopramide, metoprolol, and tetracycline. All these active ingredients either have a narrow absorption window mainly restricted to the upper GI and/or are desired to act pharmacologically directly in the stomach. In further embodiments, the active ingredient may be selected from the group comprising carbidopa, benserazide, L-DOPA, garbapentin, metformin, amoxicillin, metronidazole, clarithromycin, nitrofurantoin, acyclovir, furosemide, captopril, metoprolol, ranitidine, famotidine, ciprofloxacin, ofloxacin, verapamil, atenolol, baclofen, ciprofloxacin, cefuroxime axetil, celecoxib, diltiazem, metoclopramide, metoprolol, and tetracycline. It will be appreciated that the first and second active ingredients may be the same or may be different. Further, it will be appreciated that the first and/or second active ingredient may each be one or more active ingredients.

Non-limiting examples which embody certain aspects of the invention will now be described.

### EXAMPLES

### Example 1

### Desired Release Rate Based on Human Oral and Colonic Absolute Bioavailability of 5-HTP

### Methods

Determination of absolute and regional intestinal bioavailability of 5-HTP in humans.

5-HTP dosing: The free base form of 5-HTP was used (5-HTP has a water solubility of >10 mg/mL).

Colonic: 5-HTP free base saline solution 200 mg.

Intravenous: 5-HTP free base saline solution 50 mg.

Oral/Upper GI: Two 5-HTP gelatin tablets of 100 mg 5-HTP free base (200 mg total dose).

Subjects: Healthy male and female volunteers aged 18 to 65 years with a body mass index (BMI) of 19 to 28 were eligible for the study. Subjects were admitted to the investigational medical unit (IMU) 2 h before 5-HTP administration and remained at the IMU for 24 h following, for blood sampling and safety assessment.

Study sequence: All subjects received 5-HTP 200 mg on 3 occasions. (1) Colonic (200 mg 5-HTP solution by colonoscopy. (2) Intravenous (IV). (3) Upper GI (oral). At least 6 days must have passed between each visit.

Plasma samples analysis: Plasma samples were stored at −80° C. until analysis. 5-HTP and the metabolite 5-hydroxyindole-acetic-acid (5-HIAA) were quantified by liquid chromatography with mass-spectrometry detection.

Data analysis: The PK data were analyzed using noncompartmental (NCA) and compartmental (mixed effects) mathematical modelling approaches, to calculate area 5-HTP plasma under the curve (AUC) for each subject for each 5-HTP administration. This data was used for calculating the 5-HTP absolute bioavailability and relative upper GI tract:colon bioavailability data, according to formulas provided below.

Results: The human bioavailability of 5-HTP via the various routes of administration above was established in human subjects by administering 5-HTP through these various routes and quantifying the resultant 5-HTP plasma levels at various time points. All human subjects received 5-HTP via each of the three administration routes on separate days. Plasma samples for 5-HTP quantification were collected for 24 h at selected time periods and the results are shown in FIG. 1.

From FIG. 1, the area under the curve (AUC) for each route of administration was obtained, which was then used to calculate the absolute bioavailability (F) of the oral and colon routes of administration. For example, the formula for calculating F for a drug administered by the oral route (po) is given below (D is dose):

$${F}_{po} = 100 \% \times \frac{A U {C}_{p o} \times {D}_{i v}}{A U {C}_{iv} \times {D}_{p o}}$$

A similar formula was used to calculate the absolute bioavailability after colonic dosing. The AUC for oral dosing was 1505 (h*ng/ml), for colonic dosing it was 312 (h*ng/ml), and for intravenous dosing it was 2042 (h*ng/ml), which values were used to provide oral and colonic bioavailabilities, as shown below.

$${F}_{p o} = 100 \% \times \frac{1505 ( h * ng / ml ) \times 50 mg}{2042 ( h * n g / ml ) \times 200 mg} \approx 20 \%$$

$${F}_{c o l o n} = 100 \% \times \frac{312 ( h * ng / ml ) \times 50 mg}{2042 ( h * n g / ml ) \times 200 mg} \approx 4 \%$$

Based on the calculations above, the absolute 5-HTP bioavailability through the oral route was 20% (upper GI tract), while the absolute bioavailability of 5-HTP from colonic dosing was 4%. The relative bioavailability oral:colon was calculated as follows:

$${F}_{rel} = 100 \% \times \frac{A U {C}_{c o} \times {D}_{po}}{A U {C}_{p o} \times {D}_{c o}}$$

$$Thus : \\ {F}_{rel} = 100 \% \times \frac{312 ( h * ng / ml ) \times 200 mg}{1505 ( h * ng / ml ) \times 200 mg} \approx 20 \%$$

As calculated above, the relative upper GI tract:colon bioavailability was 20% (corresponding to an absolute colonic bioavailability of 4%). Based on the above, according to general teachings in the field of sustained-release drug delivery (Sutton S C. The use of gastrointestinal intubation studies for controlled release development. Br J Clin Pharmacol. 2009 September; 68[3]: 342-54) it is concluded that gastroretentive technologies are needed in order to achieve a desirable extended release profile of 5-HTP together with a desirable sustained and therapeutically active 5-HTP plasma level profile. Further, the determination of the oral bioavailability (Fₚₒ) enables computation of the delivery rate of 5-HTP needed from the dosage form to achieve a given plasma level of 5-HTP at steady state. Moreover, computation of the resultant plasma levels of 5-HTP can be done for embodiments where a peripheral decarboxylase inhibitor (with different levels of effects on 5-HTP bioavailability and elimination half-life) is included in the dosage form.

Release Rate Calculations:

### Scenario 1

The desired release rate profile was calculated as follows, for a dosage form disclosed in the present invention when administering 5-HTP without a peripheral decarboxylase inhibitor:

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 1.5 hrs=0.462 h⁻¹; and
F=bioavailability, ˜0.2 for 5-HTP (for oral route of administration).
   Substituting, we get Rᵢₙₚᵤₜ=˜25 mg/hr, to achieve average steady-state plasma concentrations of 5-HTP of 1 mg/L (1000 ng/ml).

### Scenario 2

The desired release rate profile was calculated as follows, for a dosage form disclosed in the present invention when administering 5-HTP with a peripheral decarboxylase inhibitor, such as carbidopa or benserazide, that increases 5-HTP bioavailability by 1-fold without substantially changing 5-HTP plasma half-life:

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 1.5 hrs=0.462 h⁻¹; and
F=bioavailability, ˜0.4 for 5-HTP (for oral route of administration).
   Substituting, we get Rᵢₙₚᵤₜ=˜12.5 mg/hr, to achieve average steady-state plasma concentrations of 5-HTP of 1 mg/L (1000 ng/ml).

### Scenario 3

The desired release rate profile was calculated as follows, for a dosage form disclosed in the present invention when administering 5-HTP with a peripheral decarboxylase inhibitor, such as carbidopa or benserazide, that increases 5-HTP's bioavailability by 1-fold and increases 5-HTP's plasma half-life to 2 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2 hrs=0.347 h⁻¹; and
F=bioavailability, ˜0.4 for 5-HTP (for oral route of administration).
   Substituting, we get Rᵢₙₚᵤₜ=˜8.7 mg/hr, to achieve average steady-state plasma concentrations of 5-HTP of 1 mg/L (1000 ng/ml).

### Scenario 4

The desired release rate profile was calculated as follows, for a dosage form disclosed in the present invention when administering 5-HTP with a peripheral decarboxylase inhibitor, such as carbidopa or benserazide, that increases 5-HTP's bioavailability by 1-fold and increases 5-HTP's plasma half-life to 2.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2.5 hrs=0.277 h; and
F=bioavailability, ˜0.4 for 5-HTP (for oral route of administration).
   Substituting, we get Rᵢₙₚᵤₜ=˜6.9 mg/hr, to achieve average steady-state plasma concentrations of 5-HTP of 1 mg/L (1000 ng/ml).

### Scenario 5

The desired release rate profile was calculated as follows, for a dosage form disclosed in the present invention when administering 5-HTP with a peripheral decarboxylase inhibitor, such as carbidopa or benserazide, that increases 5-HTP's bioavailability by 1-fold and increases 5-HTP's plasma half-life to 3 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3 hrs=0.231 h⁻¹; and
F=bioavailability, ˜0.4 for 5-HTP (for oral route of administration).
   Substituting, we get Rᵢₙₚᵤₜ=˜5.8 mg/hr, to achieve average steady-state plasma concentrations of 5-HTP of 1 mg/L (1000 ng/ml).

### Scenario 6

The desired release rate profile was calculated as follows, for a dosage form disclosed in the present invention when administering 5-HTP with a peripheral decarboxylase inhibitor, such as carbidopa or benserazide, that increases 5-HTP's bioavailability by 1-fold and increases 5-HTP's plasma half-life to 3.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3.5 hrs=0.198 h⁻¹; and
F=bioavailability, ˜0.4 for 5-HTP (for oral route of administration).
   Substituting, we get Rᵢₙₚᵤₜ=˜4.9 mg/hr, to achieve average steady-state plasma concentrations of 5-HTP of 1 mg/L (1000 ng/ml).

### Scenario 7

The desired release rate profile was calculated as follows, for a dosage form disclosed in the present invention when administering 5-HTP with a peripheral decarboxylase inhibitor, such as carbidopa or benserazide, that increases 5-HTP bioavailability by 2-fold without substantially changing 5-HTP plasma half-life:

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 1.5 hrs=0.462 h⁻¹; and
F=bioavailability, ˜0.6 for 5-HTP (for oral route of administration). Substituting, we get Rᵢₙₚᵤₜ=˜8.3 mg/hr, to achieve average steady-state plasma concentrations of 5-HTP of 1 mg/L (1000 ng/ml).

### Scenario 8

The desired release rate profile was calculated as follows, for a dosage form disclosed in the present invention when administering 5-HTP with a peripheral decarboxylase inhibitor, such as carbidopa or benserazide, that increases 5-HTP's bioavailability by 2-fold and increases 5-HTP's plasma half-life to 2 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2 hrs=0.347 h⁻¹; and
F=bioavailability, ˜0.6 for 5-HTP (for oral route of administration).
   Substituting, we get Rᵢₙₚᵤₜ=˜5.8 mg/hr, to achieve average steady-state plasma concentrations of 5-HTP of 1 mg/L (1000 ng/ml).

### Scenario 9

The desired release rate profile was calculated as follows, for a dosage form disclosed in the present invention when administering 5-HTP with a peripheral decarboxylase inhibitor, such as carbidopa or benserazide, that increases 5-HTP's bioavailability by 2-fold and increases 5-HTP's plasma half-life to 2.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2.5 hrs=0.277 h⁻¹; and
F=bioavailability, ˜0.6 for 5-HTP (for oral route of administration).
   Substituting, we get Rᵢₙₚᵤₜ=˜4.6 mg/hr, to achieve average steady-state plasma concentrations of 5-HTP of 1 mg/L (1000 ng/ml).

### Scenario 10

The desired release rate profile was calculated as follows, for a dosage form disclosed in the present invention when administering 5-HTP with a peripheral decarboxylase inhibitor, such as carbidopa or benserazide, that increases 5-HTP's bioavailability by 2-fold and increases 5-HTP's plasma half-life to 3 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3 hrs=0.231 h⁻¹; and
F=bioavailability, ˜0.6 for 5-HTP (for oral route of administration).
   Substituting, we get Rᵢₙₚᵤₜ=˜3.9 mg/hr, to achieve average steady-state plasma concentrations of 5-HTP of 1 mg/L (1000 ng/ml).

### Scenario 11

The desired release rate profile was calculated as follows, for a dosage form disclosed in the present invention when administering 5-HTP with a peripheral decarboxylase inhibitor, such as carbidopa or benserazide, that increases 5-HTP's bioavailability by 2-fold and increases 5-HTP's plasma half-life to 3.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3.5 hrs=0.198 h⁻¹; and
F=bioavailability, ˜0.6 for 5-HTP (for oral route of administration).
   Substituting, we get Rᵢₙₚᵤₜ=˜3.3 mg/hr, to achieve average steady-state plasma concentrations of 5-HTP of 1 mg/L (1000 ng/ml).

### Scenario 12

The desired release rate profile was calculated as follows, for a dosage form disclosed in the present invention when administering 5-HTP with a peripheral decarboxylase inhibitor, such as carbidopa or benserazide, that increases 5-HTP's bioavailability by 2-fold and increases 5-HTP's plasma half-life to 4 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 4 hrs=0.173 h⁻¹; and
F=bioavailability, ˜0.6 for 5-HTP (for oral route of administration).
   Substituting, we get Rᵢₙₚᵤₜ=˜2.9 mg/hr, to achieve average steady-state plasma concentrations of 5-HTP of 1 mg/L (1000 ng/ml).

### Scenario 13

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold without substantially changing 5-HTP plasma half-life:

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 1.5 hrs=0.462 h⁻¹; and
F=bioavailability, ˜0.8 for 5-HTP (for oral route of administration).
   Substituting, we get Rᵢₙₚᵤₜ=˜5.8 mg/hr, to achieve average steady-state plasma concentrations of 5-HTP of 1 mg/L (1000 ng/ml).

### Scenario 14

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold and increases 5-HTP's plasma half-life to 2 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2 hrs=0.347 h⁻¹; and
F=bioavailability, ˜0.8 for 5-HTP (for oral route of administration).
   Substituting, we get Rᵢₙₚᵤₜ=˜4.33 mg/hr, to achieve average steady-state plasma concentrations of 5-HTP of 1 mg/L (1000 ng/ml).

### Scenario 15

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold and increases 5-HTP's plasma half-life to 2.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 2.5 hrs=0.277 h⁻¹; and
F=bioavailability, ˜0.8 for 5-HTP (for oral route of administration).
   Substituting, we get Rᵢₙₚᵤₜ=˜3.5 mg/hr, to achieve average steady-state plasma concentrations of 5-HTP of 1 mg/L (1000 ng/ml).

### Scenario 16

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold and increases 5-HTP's plasma half-life to 3 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3 hrs=0.231 h⁻¹; and
F=bioavailability, ˜0.8 for 5-HTP (for oral route of administration).
   Substituting, we get Rᵢₙₚᵤₜ=˜2.9 mg/hr, to achieve average steady-state plasma concentrations of 5-HTP of 1 mg/L (1000 ng/ml).

### Scenario 17

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold and increases 5-HTP's plasma half-life to 3.5 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 3.5 hrs=0.198 h⁻¹; and
F=bioavailability, ˜0.8 for 5-HTP (for oral route of administration).
   Substituting, we get Rᵢₙₚᵤₜ=˜2.5 mg/hr, to achieve average steady-state plasma concentrations of 5-HTP of 1 mg/L (1000 ng/ml).

### Scenario 18

The desired release rate profile was calculated as follows, for a dosage form disclosed in the present invention when administering 5-HTP with a peripheral decarboxylase inhibitor, such as carbidopa or benserazide, that increases 5-HTP's bioavailability by 3-fold and increases 5-HTP's plasma half-life to 4 h.

Rᵢₙₚᵤₜ=(Cₛₛ×V_{d}×kₑₗ)/F

### Where:

Rᵢₙₚᵤₜ=release rate of 5-HTP from the particles/matrix;
Cₛₛ=steady-state plasma concentration desired (=1 mg/mL for 5-HTP);
V_{d}=volume of distribution of 5-HTP (˜10 L);
kₑₗ=elimination rate constant, hr-1, calculated from plasma half-life value of 4 hrs=0.173 h⁻¹; and
F=bioavailability, ˜0.8 for 5-HTP (for oral route of administration).
   Substituting, we get Rᵢₙₚᵤₜ=˜2.2 mg/hr, to achieve average steady-state plasma concentrations of 5-HTP of 1 mg/L (1000 ng/ml).

It is understood within the art that determining F will enable calculation of the required delivery rate (R_{Input}) to achieve a given steady-state plasma concentration (Cₛₛ), and the above Scenarios exemplify calculated relationships within the scope of the instant invention, which also includes cases falling between, above, and below the exemplified input parameters. It is further understood that values deviating minimally from the exemplified will have no functional consequences and are hence encompassed within the instant invention.

For steady-state plasma concentration (C %₅) above and below the in the Scenarios exemplified, e.g. 100 ng/ml to 3000 ng/ml, it is understood from the equation

Rᵢₙₚᵤₜ=(Cₛₛ×V_{d}×kₑₗ)/F

that the required delivery rate (R_{Input}) can simply be increased or decreased proportionally, e.g. double the required delivery rate to obtain double the steady-state plasma concentrations.

It is known in the art that a peripheral decarboxylase inhibitor enhances 5-HTP's bioavailability in a manner dependent on the dose and regimen (Gijsman H J, van Gerven J M, de Kam M L, Schoemaker R C, Pieters M S, Weemaes M, de Rijk R, van der Post J, Cohen A F. “Placebo-controlled comparison of three dose-regimens of 5-hydroxytryptophan challenge test in healthy volunteers.” J Clin Psychopharmacol. (2002), 22(2):183-9. PubMed PMID: 11910264; Westenberg H G, Gerritsen T W, Meijer B A, van Praag H M. “Kinetics of l-5-hydroxytryptophan in healthy subjects.” Psychiatry Res. (1982), 7(3):373-85. PubMed PMID: 6187038). To determine a dosing-regimen of a peripheral decarboxylase inhibitor that will enhance 5-HTP bioavailability 1-fold to 3-fold, the dose of peripheral decarboxylase inhibitor can simply be adjusted upward until the desired F is obtained.

Two representative clinically used peripheral decarboxylase inhibitors are carbidopa and benserazide. When used with levodopa to treat Parkinson's disease, the usual levodopa:carbidopa or levodopa:benserazide ratio is 4:1 and the absolute clinical dosage levels of carbidopa and benserazide are similar. Thus, carbidopa and benserazide dosage levels are functionally interchangeable.

In some embodiments, a dose of a peripheral decarboxylase inhibitor that would enhance 5-HTP bioavailability 1-fold to 2-fold would be <1 mg/kg/day, in some embodiments in the range of 0.1 to 0.5 mg/kg/day. In some embodiments, a dose of a peripheral decarboxylase inhibitor that would enhance 5-HTP bioavailability about 2-fold would be ˜2 mg/kg/day, in some embodiments in the range of 1 to 2 mg/kg/day. In some embodiments, a dose of a peripheral decarboxylase inhibitor that would enhance 5-HTP bioavailability about 3-fold would be >2 mg/kg/day, in some embodiments in the range of 2 to 2.5 mg/kg/day.

Ideally, the release rate profile is linear or substantially linear over the 12 hour period, such that a sufficient amount of 5-HTP is released every hour to maintain the desired steady state concentration in the body.

### Example 2

### Dual Swellable System

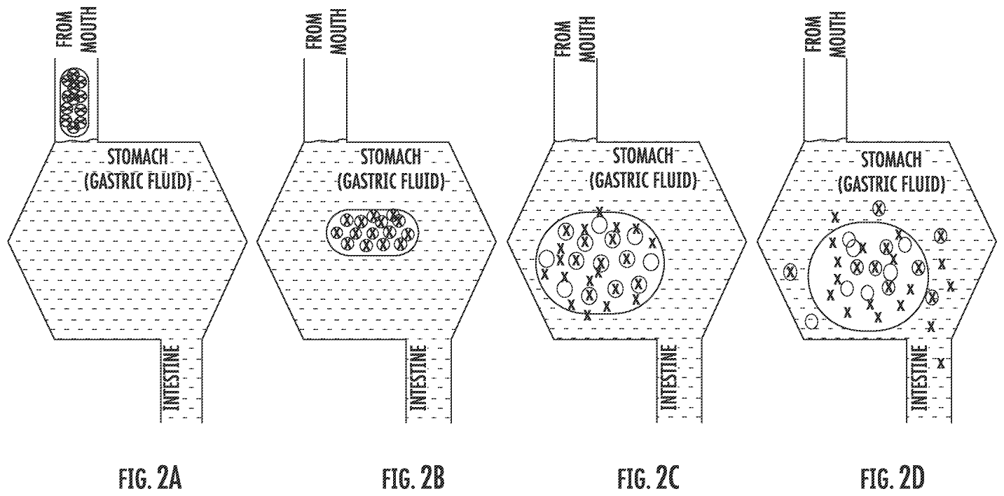

A dual swellable system is proposed as one exemplary way to achieve the desired release profile. In this system, the 5-HTP is formulated as part of a microparticle, which may be swellable or not. The resulting microparticles are then placed within a swellable polymeric matrix, which also contains 5-HTP, to form the dosage form. As illustrated in FIGS. 2A and 2B, following administration and once the dosage form reaches the stomach, the swellable matrix surrounding the microparticles swells up and prevents the dosage form from exiting the stomach. Thus, the 5-HTP, and other included active ingredients, contained in the microparticles, is initially released from the microparticles into the polymeric matrix first (see FIG. 2C), followed by diffusion of the drug through the matrix into the gastric fluid. See FIG. 2D. However, it is possible that some fraction of the microparticles also efflux with the 5-HTP and hence release 5-HTP, and other included active ingredients, directly into the gastric fluids. See FIG. 2D. Any 5-HTP, or other included active ingredients, that is directly contained in the swellable matrix diffuses through the matrix out into the gastric fluid.

### Microparticles Containing 5-HTP:

Microparticles of 5-HTP may be made using:
gelatin crosslinked by a chemical crosslinking agent (e.g. genipin);
gelatin-PEGDA (gelatin-polyethylene glycol diacrylate) crosslinked by UV light;
PEGDA crosslinked by UV light;
sodium acrylate crosslinked with a metal ion having a charge of more than 1+;
crosslinked or non-crosslinked chitosan having a degree of deacetylation of from 20 to 70%; or
non-crosslinked poly(ethylene oxide) and/or hydroxypropyl methylcellulose.

The 5-HTP may be provided in an amount of from 1 to 50 wt % of the weight of the microparticles. Microparticles may also be made without cross-linking, by mixing polymers and excipients, using standard methods well-understood by a practioner in the art. See e.g. U.S. Pat. Nos. 6,475,521; and 7,094,427.

Microparticles containing 5-HTP, and other included active ingredients, may be made using a number of conventional techniques, including: spray-drying a solution of matrix and 5-HTP and/or other active ingredients; water and oil emulsion methods; precipitation under agitation; and the like. These microparticles may be crosslinked using different methodologies depending on the polymer used.

For example, the microparticles may be prepared by the following methods. First, a simple oil-in-water emulsion method, where the non-crosslinked polymer, 5-HTP, other active ingredients, and excipients will be dissolved in water and then emulsified into an organic solvent. The solvent is then evaporated off, and, if necessary, the residue is crosslinked (e.g. by UV or chemical means) and then lyophilized to form the desired microparticles.

In a second exemplary method, a multilamellar liposome is used as a template. In this case, liposomes are formed by drying a solution of lipids into a film form, then hydrating this with an aqueous solution consisting of the crosslinkable polymer, 5-HTP, other active ingredients, and excipients. The resulting liposome is then crosslinked and dialyzed to remove non-crosslinked material. The lipid layer is then stripped off with detergent to yield the desired gel particles.

The microparticles made using the above techniques are designed to exhibit differential swelling at different pH values. This swelling will provide slow-release of 5-HTP and other active ingredients from the particles, which can be studied (in the microparticles alone) using state-of-the-art dialysis techniques. Relevant variables to consider include crosslink density and the loading amount of 5-HTP.

### Crosslinking of Polymeric Materials:

As will be noted hereinbefore, both the microparticles and the polymeric matrix that encapsulates these microparticles may be in the form of a crosslinked material. Such crosslinkable materials may be formed from suitable aqueous formulations of the non-crosslinked polymeric materials, which include:
gelatin in an amount of from 1 to 20 wt % in water (where crosslinking is performed by the addition of less than 1 wt % of genipin at an appropriate stage);
gelatin-PEGDA in an amount of from 1 to 20 wt % in water (where crosslinking is performed by the addition of from 0.05 to 0.5 wt % of Irgacure 2959 at an appropriate stage);
PEGDA or other polyacrylic acids in an amount of from 1 to 20 wt % in water (where crosslinking is performed by the addition of from 0.05 to 0.5 wt % of Irgacure 2959 at an appropriate stage);
sodium acrylate in an amount of from 0.5 to 10 wt % in water (where crosslinking is performed by the addition of from 0.1 to 1.0 wt % of an appropriate metal salt at an appropriate stage);
chitosan having a degree of deacetylation of from 20 to 70% (where crosslinking is performed by coacervation using epichlorhydrin or glutaraldehyde; where excess crosslinker is washed off);
crosslinked hyaluronic acid (crosslinked by any suitable means known to the person skilled in the art, such as by chemical crosslinking or by UV crosslinking of a hyaluronic acid polymer having methacrylate groups (e.g. by UV crosslinking of the methacrylate groups);
crosslinked hydroxyl propyl cellulose (crosslinked by any suitable means known to the person skilled in the art); and
crosslinked hydroxyl propyl methyl cellulose (crosslinked by any suitable means known to the person skilled in the art).

### Polymeric Matrix:

The above microparticles will be held within a capsule formed by a crosslinked gelatin-PEGDA matrix (the crosslinking step is conducted in a capsule mold) or within a crosslinked gelatin-PEGDA matrix within a gelatin capsule. Other materials that may be used include, crosslinked hyaluronic acid, crosslinked chitosan, crosslinked hydroxyl propyl cellulose, crosslinked hydroxyl propyl methyl cellulose, and crosslinked sodium acrylate.

The microparticles may be dispersed in a crosslinkable hydrogel matrix, and then the matrix is crosslinked. This matrix material may be in the form of a filled capsule. The capsule will swell to a sphere that will be retained in the stomach.

The effects of crosslink density on the capsule swelling dimensions are studied in a simulated gastric fluid at pH 1.5 to 3.5 (normal gastric pH range) and at a pH of 6.5 (simulating the small intestine).

As will be appreciated, 5-HTP may also form part of the swellable matrix, though this is optional (e.g. it can be present in an amount of from 0 to 50 wt %). As such, when 5-HTP is present in the swellable matrix (i.e. the first polymeric matrix material), it may be provided in an amount of from 1 to 50 wt % (e.g. from 1 to 45 wt %) of the weight of said swellable matrix material. If other active ingredients are included, these ranges apply to the total active ingredient content.

Resulting Dosage Form:

As an example of a full dosage form according to the above, microparticles of a gelatin-PEGDA hydrogel containing 5-HTP (20 wt %) may be dispersed within a capsule made from a gelatin-PEGDA matrix. This capsule is designed to swell to a sphere of sufficiently large dimensions upon contact with gastric fluid so that it will be retained in the stomach, such that the 5-HTP is released in the manner discussed hereinbefore and will provide the desired target release rate of ˜25 mg/hr.

The microparticles can to a substantial degree be released from the swellable matrix and to a substantial degree release 5-HTP and other active ingredients in the upper GI after exiting the swellable matrix. Alternatively, the microparticles will be mainly retained in the swellable matrix while delivering 5-HTP and other active ingredients, and 5-HTP and other active ingredients will diffuse out through the swellable matrix.

### Example 3

### Swellable Tablet Too Large to Pass Through the Pyloric Sphincter

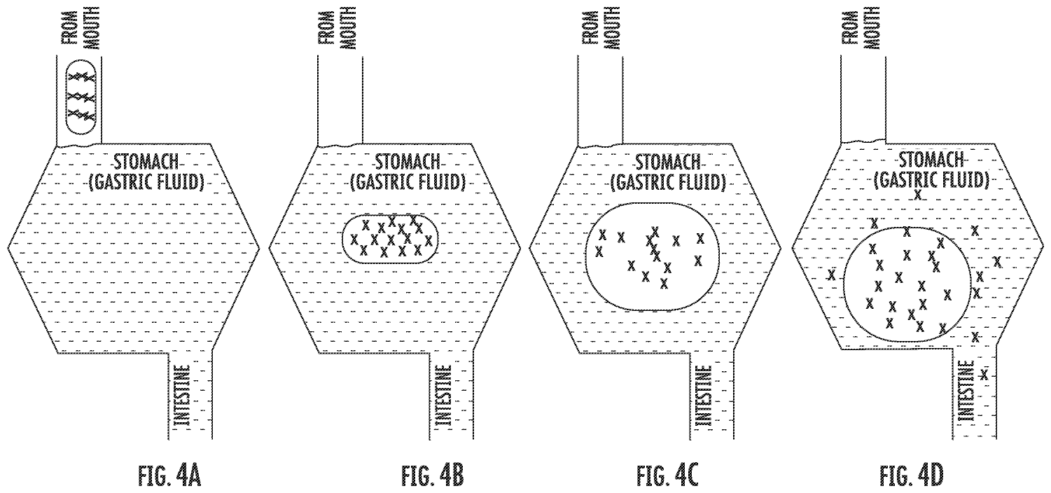

A swellable tablet will be prepared in accordance with those discussed in U.S. Pat. Nos. 6,340,475; 6,635,280; and 7,438,927, the contents of which are incorporated herein by reference, except that the main active compound used herein is 5-HTP. In some embodiments, the swellable tablet can be as shown in FIGS. 4A-4D.

As discussed in the above-referenced US patents, the swellable tablets disclosed therein swell in the stomach into a sphere that is too large to pass through the pyloric sphincter (and hence out of the stomach). Thus, the tablet is retained in the stomach for up to 12 hours, during which time 5-HTP is slowly released for eventual absorption in the upper GI.

Suitable gastroretentive 5-HTP formulations (GR1, GR2, and GR3) may be manufactured using a standard granulation technique with the ingredients set forth in Table 1 below.

TABLE 1
Exemplary Gastroretentive 5-HTP Formulations.

| Ingredient | GR1 | | GR2 | | GR3 | |
|---|---|---|---|---|---|---|
| 5-HTP | 300 | mg | 300 | mg | 600 | mg |
| | (44.76 wt %) | | (44.76 wt %) | | (61.11 wt %) | |
| METHOCEL ™ K15M, | 21.99 | wt % | | | 7.59 | wt % |
| premium | | | | | | |
| METHOCEL ™ K4M, | | | 16.46 | wt % | | |
| premium | | | | | | |
| SENTRY ™ POLYOX ™ | 21.99 | wt % | | | | |
| WSR Coagulant, NF FP | | | | | | |
| SENTRY ™ POLYOX ™ | | | 21.99 | wt % | 27.09 | wt % |
| WSR 303, NF FP | | | | | | |
| AVICEL ™ PH-101 NF | 7.49 | wt % | 12.98 | wt % | 0.00 | wt % |
| METHOCEL ™ E5, premium | 2.75 | wt % | 2.75 | wt % | 3.22 | wt % |
| Magnesium stearate, NF | 1.00 | wt % | 1.00 | wt % | 1.00 | wt % |
| Tablet Weight | 670 | mg | 670 | mg | 982 | mg |

| | | | |
|---|---|---|---|
| Tablet dimensions/form | 0.3937″ × | 0.3937″ × | 0.4062″ × |
| | 0.6299″ | 0.6299″ | 0.75″ |
| | (1 cm × 1.6 | (1 cm × 1.6 | (1.032 cm × 1.905 |
| | cm)/Mod Oval | cm)/Mod Oval | cm)/Mod Cap |

Cellulose ethers sold under the tradename METHOCEL™ (Dow Chemical Company, Midland, Mich., United States of America) comprise hydroxypropyl methylcellulose (also known as hypromellose), and water-soluble resins sold under the tradename SENTRY™ POLYOX™ (Dow Chemical Company, Midland, Mich., United States of America) comprise polyethylene oxide. METHOCEL™ E5, premium is a USP type 2910 hydroxypropyl methylcellulose with number average molecular weight of on the order of 6000-8000 and a viscosity of 5 cps as a 2% aqueous solution at 20° C. METHOCEL™ K4M and METHOCEL™ K15M are USP type 2208 hydroxypropyl methylcellulose with viscosities of 4000 cps and 15,000 cps, respectively, as a 2% aqueous solution at 20° C., and number average molecular weights of the order of 80,000 and 100,000, respectively. SENTRY™ POLYOX™ WSR 301, NF FP, SENTRY™ POLYOX™ WSR Coagulant, NF FP and SENTRY™ POLYOX™ WSR 303, NF FP have viscosity-average molecular weights of approximately 4,000,000, 5,000,000 and 7,000,000, respectively. Cellulose sold under the tradename AVICEL™ (PMC Corporation, Philadelphia, Pa., United States of America) PH-101, NF is microcrystalline cellulose. The polymers, e.g. polyethylene oxide or methylcellulose, are usually not cross-linked.

Formulation in this manner may allow for once- or twice-daily dosing of 5-HTP with a linear release profile (i.e., wherein a graph of the total amount of 5-HTP released versus time is substantially linear). This is based, at least in part, on the fact that the active compound gabapentin (the active compound of U.S. Pat. No. 7,438,927), has a similar molecular weight and physiochemical properties to 5-HTP.

In some embodiments, the swellable solid dosage form can comprise more than one compartment. In some embodiments, a first compartment contains 5-HTP while a second compartment contains a second active ingredients, such as a peripheral decarboxylase inhibitor or a serotonin enhancing compound. Analogous to the foregoing, in some embodiments the solid dosage form comprises three or more compartments, carrying different active ingredients or providing different release profiles. In some embodiments, one compartment primarily provides the gastroretentive element.

In some embodiments, the dosage form includes a coating. In some embodiments a smooth coating facilitates swallowing, in other embodiments a coating masks an unpalatable taste, in yet other embodiments a coating serves an aesthetic function, and in yet other embodiments a coating protects the physical or chemical integrity of the dosage form. In some embodiments, the coating carries an active ingredient, which in some embodiments can be a serotonin-enhancing drug, for instance, but not limited to, a serotonin reuptake inhibitor. Further, a coating can serve more than one purpose. Solid dosage form coatings for the aforementioned purposes are well-known in the art.

### Example 4

### Swellable Tablet Too Large to Pass Through the Pyloric Sphincter Incorporating Microparticles

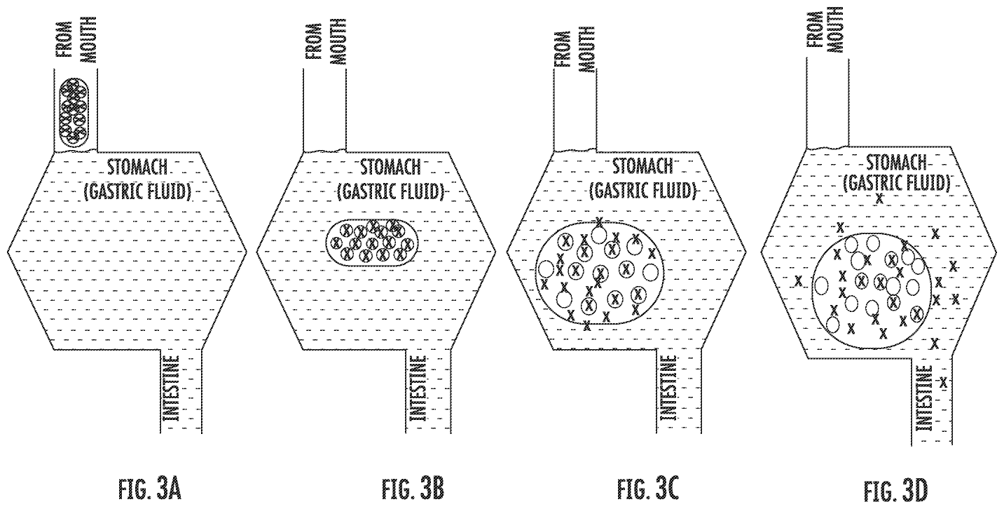

Swellable tablets will be prepared in accordance with Example 3, with the addition that microparticles are incorporated into the matrix, in accordance with methods discussed in U.S. Pat. No. 6,475,521, incorporated herein by reference in its entirety. The microparticles containing 5-HTP, and optional other active ingredients, are dispersed within the matrix. The 5-HTP, and optional other active ingredients, are released over time from the microparticles, either by diffusion, erosion, or both. The 5-HTP, and optional other active ingredients, diffuse though the matrix to the gastric fluid and therefrom to the upper intestine where absorption occurs. See FIGS. 3A-3D.

### Example 5

### Push-Pull Osmotic Pump

A push-pull osmotic pump will be prepared where the external dimensions are greater than 1 cm in at least 2 dimensions based on U.S. Pat. No. 4,765,989, incorporated herein by reference in its entirety, except including 5-HTP as an active ingredient. The pump is coated with a cellulose acetate or other water permeable, but drug impermeable membrane. The core comprises a swelling agent such as polyethylene oxide in one layer and a separate drug with an osmotic agent in the second layer. The second layer is in contact with that portion of the semipermeable membrane through which a hole or holes are fabricated or designed to appear after dosing. The benefit of the dosage form is a constant release profile for both drugs or other patterns of release profile. The drug delivery time should be designed to be no greater than 9 hours and no shorter than 5 hours.

### REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
Birdsall T C. “5-Hydroxytryptophan: a clinically-effective serotonin precursor.” Altern Med Rev. (1998), 3(4):271-80. Review. PubMed PMID: 9727088.
Blier, Pierre, and Claude De Montigny. “Current advances and trends in the treatment of depression.” Trends in pharmacological sciences (1994), 15(7): 220-226.
Bryn et al., Solid-State Chemistry of Drugs, Second Edition, published by SSCI, Inc of West Lafayette, Ind., USA, 1999, ISBN 0-967-06710-3.
European Patent No. 2,120,887.
Gijsman H J, van Gerven J M, de Kam M L, Schoemaker R C, Pieters M S, Weemaes M, de Rijk R, van der Post J, Cohen A F. “Placebo-controlled comparison of three dose-regimens of 5-hydroxytryptophan challenge test in healthy volunteers.” J Clin Psychopharmacol. (2002), 22(2):183-9. PubMed PMID: 11910264.
Hua S, Marks E, Schneider J J, Keely S. “Advances in oral nano-delivery systems for colon targeted drug delivery in inflammatory bowel disease: selective targeting to diseased versus healthy tissue.” Nanomedicine. (2015), 11(5):1117-32.
Jacobsen, Jacob P R, et al. “Adjunctive 5-Hydroxytryptophan slow-release for treatment-resistant depression: clinical and preclinical rationale.” Trends in pharmacological sciences (2016), 37(11): 933-944.
Jacobsen, J. P., et al., “SSRI Augmentation by 5-Hydroxytryptophan Slow Release: Mouse Pharmacodynamic Proof of Concept.” Neuropsychopharmacology (2016), 41(9):2324-34.
Remington The Science and Practice of Pharmacy, 19th ed., Mack Printing Company, Easton, Pa. (1995).
Sutton S C. “The use of gastrointestinal intubation studies for controlled release development.” Br J Clin Pharmacol. (2009), 68(3):342-54.
Timmermans, Moes, A J. J. Pharm. Sci. (1994), 83:18-24.
Turner, Erick H., Jennifer M. Loftis, and Aaron D. Blackwell. “Serotonin a la carte: supplementation with the serotonin precursor 5-hydroxytryptophan.” Pharmacology & therapeutics (2006), 109(3): 325-338.
U.S. Patent Application Publication No. 2017/0266112.
U.S. Patent Application Publication No. 2018/0311154.
U.S. Pat. No. 4,140,755.
U.S. Pat. No. 7,765,989.
U.S. Pat. No. 4,996,058.
U.S. Pat. No. 6,340,475.
U.S. Pat. No. 6,475,521.
U.S. Pat. No. 6,488,962.
U.S. Pat. No. 6,635,280.
U.S. Pat. No. 6,960,356.
U.S. Pat. No. 7,094,427.
U.S. Pat. No. 7,438,927.
U.S. Pat. No. 7,670,619.
U.S. Pat. No. 8,771,730.
U.S. Pat. No. 8,969,400.
U.S. Pat. No. 9,161,911.
Westenberg H G, Gerritsen T W, Meijer B A, van Praag H M. “Kinetics of l-5-hydroxytryptophan in healthy subjects.” Psychiatry Res. (1982), 7(3):373-85. PubMed PMID: 6187038.

It will be understood that various details of the presently disclosed subject matter may be changed without departing from the scope of the presently disclosed subject matter. Furthermore, the foregoing description is for the purpose of illustration only, and not for the purpose of limitation.

1. A gastroretentive sustained release (SR) dosage form comprising 5-hydroxytryptophan (5-HTP) or a pharmaceutically acceptable salt or solvate thereof and a pharmaceutically acceptable carrier and/or excipient, wherein the dosage form provides a release rate to the upper gastrointestinal tract of between about 2.5 milligrams per hour (mg/hr) and about 75 mg/hr, thereby providing a steady state plasma level of between about 0.1 milligrams per liter (mg/L) to about 4 mg/L at steady state.

2. The dosage form of claim 1, wherein the dosage form comprises at least a first polymeric matrix material that swells in the presence of gastric fluid, thereby providing a swellable dosage form that increases in size to promote retention of the dosage form in the stomach, optionally wherein the dosage form swells in the presence of gastric fluid to at least about 150% compared to a pre-swelling volume of the dosage form.

3. The dosage form of claim 2, wherein the first polymeric matrix material comprises a hydrophilic polymer selected from the group consisting of polyoxyethylene oxide, hydroxyethylcellulose, carboxymethylcellulose, polyethylene glycol diacrylate (PEGDA), gelatin, gelatin-PEGDA copolymer, hyaluronic acid, chitosan, hydroxypropylcellulose, hydroxypropylmethylcellulose, sodium acrylate, and copolymers thereof.

4. The dosage form of claim 2, wherein the 5-HTP or pharmaceutically acceptable salt or solvate thereof is directly dispersed in the first polymeric matrix material in an amount between about 1 weight % (wt %) and about 50 wt % based on the weight of the first polymeric matrix material.

5. The dosage form of claim 2, wherein the dosage form further comprises:
a plurality of microparticles dispersed within the first polymeric matrix material, wherein each of said microparticles comprises a second polymeric matrix material and 5-HTP or a pharmaceutically acceptable salt or solvate thereof dispersed within the second polymeric matrix material, and wherein the first polymeric matrix material comprises 5-HTP or a pharmaceutically acceptable salt or solvate thereof directly dispersed in the first polymeric matrix material in an amount between about 0 wt % and about 50 wt % based on the weight of the first polymeric matrix material.

6. The dosage form of claim 5, wherein the second polymeric matrix material comprises:
a crosslinked polymeric matrix material comprising one or more hydrophilic polymer selected from the group consisting of hydroxyl propyl methyl cellulose, hydroxyl propyl cellulose, hyaluronic acid, chitosan, gelatin, gelatin-PEGDA, PEGDA, and sodium acrylate; and/or
a non-crosslinked polymeric matrix material comprising one or more hydrophilic polymer selected from the group consisting of chitosan, poly(ethylene oxide), hydroxyl propyl cellulose and hydroxypropyl methylcellulose.

7. The dosage form of claim 5, wherein the first polymeric matrix material contains between about 5 wt % and about 50 wt % of the microparticles.

8. The dosage form of claim 5, wherein each microparticle comprises between about 1 wt % and about 30 wt % of 5-HTP or a pharmaceutically acceptable salt or solvate thereof based on the weight of the microparticle.

9. The dosage form of claim 1, wherein the dosage form comprises between about 50 milligrams (mg) and about 1,800 mg of 5-HTP or a pharmaceutically acceptable salt or solvent thereof.

10. The dosage form of claim 1, wherein at least about 30 weight % (wt %) of the 5-HTP or pharmaceutically acceptable salt or solvate thereof is released within about 4 hours of oral administration, optionally wherein at least about 50 wt % of the 5-HTP or pharmaceutically acceptable salt or solvate thereof is released within about 4 to about 9 hours of oral administration.

11. The dosage form of claim 1, wherein the dosage form further comprises one or more additional agent selected from the group consisting of a serotonin-enhancing compound, a peripheral decarboxylase inhibitor, and a gas swelling agent.

12. The dosage form of claim 1, wherein the dosage form is adapted to deliver a release profile of between about 1 mg/hr and about 42 mg/hr of 5-HTP for a period of about 12 hours, optionally wherein the release profile is substantially linear.

13. The dosage form of claim 1, the dosage form provides a release rate to the upper gastrointestinal tract of about 6.25 mg/hr, so as to provide an average steady state 5-HTP plasma level of about 0.25 mg/L.

14. A method of treating a condition selected from the group consisting of depression, social anxiety, panic disorder, generalized anxiety disorder, OCD, impulse control disorders, suicidality, borderline personality disorder, fibromyalgia, ataxia, mood symptoms and agitation related to neurological disorders, stroke recovery, autism, migraine, sleep disorders, premenstrual dysphoria, post-traumatic stress disorder, post-partum depression, phenylketonuria, and depression after interferon treatment in a patient in need of such treatment, comprising administering a dosage form of claim 1.

15. The method of claim 14, wherein the dosage form is administered once or twice daily.

16. The method of claim 14, wherein the dosage form is administered with a meal.

17. The method of claim 14, wherein the dosage form is administered once or twice daily and the total amount of 5-HTP in the daily dosage is between about 50 mg and about 3600 mg.

18. The method of claim 14, wherein the dosage form is adapted to deliver a release profile of between about 4 mg/hr and about 42 mg/hr of 5-HTP for a period of about 12 hours, optionally wherein the release profile is substantially linear.

19. The method of claim 14, wherein administration of the dosage form provides a steady state 5-HTP plasma level of between about 0.1 mg/L and about 0.9 mg/L.

20. The method of claim 14, further comprising concomitant administration of a 5-HTP absorption enhancer to increase the steady state 5-HTP plasma level between about 1-fold and about 4-fold as compared to when the 5-HTP is administered without the absorption enhancer, optionally wherein the 5-HTP absorption enhancer is a peripheral decarboxylase inhibitor.

21. A method of achieving a steady state 5-HTP plasma level of between about 0.1 mg/L to 1 mg/L, wherein the method comprises administering between about 2.5 mg/hr and about 25 mg/hr of 5-HTP or a pharmaceutically acceptable salt or solvate thereof to the upper gastrointestinal tract.

22. The method of claim 21, wherein the method achieves a steady state 5-HTP plasma level of about 0.25 mg/L by administering about 6.25 mg/hr of 5-HTP or a pharmaceutically acceptable salt or solvate thereof to the upper gastrointestinal tract.
